SEQUENCE LISTING (75 sequences)

SEQ ID NO: 1 - protein, 5 aa, Artificial sequence
  Synthetic peptide
  MISC_FEATURE (3)..(4): Xaa denotes Arg, Lys or His
HEXXH

SEQ ID NO: 2 - protein, 7 aa, Artificial sequence
  Synthetic peptide
SNYLYDN

SEQ ID NO: 3 - protein, 5 aa, Artificial sequence
  Synthetic peptide
  MISC_FEATURE (3)..(3): Xaa denotes Arg, Lys, His, Asp or Glu
SHXGY

SEQ ID NO: 4 - protein, 14 aa, Artificial sequence
  Synthetic peptide
RKFHEKHHSHRGYR

SEQ ID NO: 5 - protein, 12 aa, Artificial sequence
  Synthetic peptide
AKRHHGYKRKFH

SEQ ID NO: 6 - protein, 38 aa, Homo sapiens
  MOD_RES (2)..(2): PHOSPHORYLATION
DSHEKRHHGYRRKFHEKHHSHREFPFYGDYGSNYLYDN

SEQ ID NO: 7 - protein, 27 aa, Homo sapiens
RKFHEKHHSHREFPFYGDYGSNYLYDN

SEQ ID NO: 8 - protein, 32 aa, Homo sapiens
DSHAKRHHGYKRKFHEKHHSHRGYRSNYLYDN

SEQ ID NO: 9 - protein, 20 aa, Homo sapiens
KFHEKHHSHRGYRSNYLYDN

SEQ ID NO: 10 - protein, 24 aa, Homo sapiens
DSHAKRHHGYKRKFHEKHHSHRGY

SEQ ID NO: 11 - protein, 25 aa, Homo sapiens
DSHAKRHHGYKRKFHEKHHSHRGYR

SEQ ID NO: 12 - protein, 13 aa, Homo sapiens
RKFHEKHHSHRGY

SEQ ID NO: 13 - protein, 12 aa, Homo sapiens
KFHEKHHSHRGY

SEQ ID NO: 14 - protein, 14 aa, Homo sapiens
RKFHEKHHSHRGYR

SEQ ID NO: 15 - protein, 13 aa, Homo sapiens
KFHEKHHSHRGYR

SEQ ID NO: 16 - protein, 8 aa, Homo sapiens
KRHHGYKR

SEQ ID NO: 17 - protein, 7 aa, Homo sapiens
KRHHGYK

SEQ ID NO: 18 - protein, 5 aa, Artificial sequence
  Synthetic peptide
HEKKH

SEQ ID NO: 19 - protein, 5 aa, Artificial sequence
  Synthetic peptide
HEKRH

SEQ ID NO: 20 - protein, 5 aa, Artificial sequence
  Synthetic peptide
HEKHH

SEQ ID NO: 21 - protein, 5 aa, Artificial sequence
  Synthetic peptide
HERKH

SEQ ID NO: 22 - protein, 5 aa, Artificial sequence
  Synthetic peptide
HERRH

SEQ ID NO: 23 - protein, 5 aa, Artificial sequence
  Synthetic peptide
HERHH

SEQ ID NO: 24 - protein, 5 aa, Artificial sequence
  Synthetic peptide
HEHKH

SEQ ID NO: 25 - protein, 5 aa, Artificial sequence
  Synthetic peptide
HEHRH

SEQ ID NO: 26 - protein, 5 aa, Artificial sequence
  Synthetic peptide
HEHHH

SEQ ID NO: 27 - protein, 14 aa, Artificial sequence
  Synthetic peptide
GYKRKFHEKHHSHR

SEQ ID NO: 28 - protein, 12 aa, Artificial sequence
  Synthetic peptide
KRKFHEKHHSHR

SEQ ID NO: 29 - protein, 8 aa, Artificial sequence
  Synthetic peptide
HEKHHSHR

SEQ ID NO: 30 - protein, 6 aa, Artificial sequence
  Synthetic peptide
HEKRHH

SEQ ID NO: 31 - protein, 5 aa, Artificial sequence
  Synthetic peptide
SHRGY

SEQ ID NO: 32 - protein, 5 aa, Artificial sequence
  Synthetic peptide
SHDGY

SEQ ID NO: 33 - protein, 5 aa, Artificial sequence
  Synthetic peptide
SHKGY

SEQ ID NO: 34 - protein, 5 aa, Artificial sequence
  Synthetic peptide
SHHGY

SEQ ID NO: 35 - protein, 5 aa, Artificial sequence
  Synthetic peptide
SHEGY

SEQ ID NO: 36 - protein, 10 aa, Artificial sequence
  Synthetic peptide
HHSHRGYRSN

SEQ ID NO: 37 - protein, 10 aa, Artificial sequence
  Synthetic peptide
HEKHHSHRGY

SEQ ID NO: 38 - protein, 10 aa, Artificial sequence
  Synthetic peptide
EKHHSHRGYR

SEQ ID NO: 39 - protein, 8 aa, Artificial sequence
  Synthetic peptide
KHHSHRGY

SEQ ID NO: 40 - protein, 7 aa, Artificial sequence
  Synthetic peptide
HHSHRGY

SEQ ID NO: 41 - protein, 6 aa, Artificial sequence
  Synthetic peptide
HSHRGY

SEQ ID NO: 42 - protein, 12 aa, Artificial sequence
  Synthetic peptide
YGDYGSNYLYDN

SEQ ID NO: 43 - protein, 25 aa, Artificial sequence
  Synthetic peptide
  MISC_FEATURE (6)..(9): Xaa is present or absent and when present denotes Gly
  MISC_FEATURE (10)..(10): Xaa is present or absent and when present denotes Ser
  MISC_FEATURE (11)..(14): Xaa is present or absent and when present denotes Gly
  MISC_FEATURE (15)..(15): Xaa is present or absent and when present denotes Ser
  MISC_FEATURE (16)..(19): Xaa is present or absent and when present denotes Gly
  MISC_FEATURE (20)..(20): Xaa is present or absent and when present denotes Ser
  MISC_FEATURE (21)..(24): Xaa is present or absent and when present denotes Gly
  MISC_FEATURE (25)..(25): Xaa is present or absent and when present denotes Ser
GGGGSXXXXXXXXXXXXXXXXXXXX

SEQ ID NO: 44 - protein, 18 aa, Artificial sequence
  Synthetic peptide
KESGSVSSEQLAQFRSLD

SEQ ID NO: 45 - protein, 14 aa, Artificial sequence
  Synthetic peptide
EGKSSGSGSESKST

SEQ ID NO: 46 - protein, 8 aa, Artificial sequence
  Synthetic peptide
GGGGGGGG

SEQ ID NO: 47 - protein, 12 aa, Artificial sequence
  Synthetic peptide
GSAGSAAGSGEF

SEQ ID NO: 48 - protein, 20 aa, Artificial sequence
  Synthetic peptide
  MISC_FEATURE (5)..(6): Xaa is present or absent and when present denotes Gly
  MISC_FEATURE (7)..(7): Xaa is present or absent and when present denotes Ser
  MISC_FEATURE (8)..(10): Xaa is present or absent and when present denotes Gly
  MISC_FEATURE (11)..(11): Xaa is present or absent and when present denotes Ser
  MISC_FEATURE (12)..(14): Xaa is present or absent and when present denotes Gly
  MISC_FEATURE (15)..(15): Xaa is present or absent and when present denotes Ser
  MISC_FEATURE (16)..(18): Xaa is present or absent and when present denotes Gly
  MISC_FEATURE (19)..(19): Xaa is present or absent and when present denotes Ser
  MISC_FEATURE (20)..(20): Xaa is present or absent and when present denotes Gly
GGSGXXXXXXXXXXXXXXXX

SEQ ID NO: 49 - protein, 10 aa, Artificial sequence
  Synthetic peptide
  MISC_FEATURE (3)..(3): Xaa is present or absent and when present denotes Gly
  MISC_FEATURE (4)..(4): Xaa is present or absent and when present denotes Ser
  MISC_FEATURE (5)..(5): Xaa is present or absent and when present denotes Gly
  MISC_FEATURE (6)..(6): Xaa is present or absent and when present denotes Ser
  MISC_FEATURE (7)..(7): Xaa is present or absent and when present denotes Gly
  MISC_FEATURE (8)..(8): Xaa is present or absent and when present denotes Ser
  MISC_FEATURE (9)..(9): Xaa is present or absent and when present denotes Gly
  MISC_FEATURE (10)..(10): Xaa is present or absent and when present denotes Ser
GSXXXXXXXX

SEQ ID NO: 50 - protein, 25 aa, Artificial sequence
  Synthetic peptide
  MISC_FEATURE (6)..(6): Xaa is present or absent and when present denotes Glu
  MISC_FEATURE (7)..(9): Xaa is present or absent and when present denotes Ala
  MISC_FEATURE (10)..(10): Xaa is present or absent and when present denotes Lys
  MISC_FEATURE (11)..(11): Xaa is present or absent and when present denotes Glu
  MISC_FEATURE (12)..(14): Xaa is present or absent and when present denotes Ala
  MISC_FEATURE (15)..(15): Xaa is present or absent and when present denotes Lys
  MISC_FEATURE (16)..(16): Xaa is present or absent and when present denotes Glu
  MISC_FEATURE (17)..(19): Xaa is present or absent and when present denotes Ala
  MISC_FEATURE (20)..(20): Xaa is present or absent and when present denotes Lys
  MISC_FEATURE (21)..(21): Xaa is present or absent and when present denotes Glu
  MISC_FEATURE (22)..(24): Xaa is present or absent and when present denotes Ala
  MISC_FEATURE (25)..(25): Xaa is present or absent and when present denotes Lys
EAAAKXXXXXXXXXXXXXXXXXXXX

SEQ ID NO: 51 - protein, 27 aa, Artificial sequence
  Synthetic peptide
  MISC_FEATURE (7)..(7): Xaa is present or absent and when present denotes Glu
  MISC_FEATURE (8)..(10): Xaa is present or absent and when present denotes Ala
  MISC_FEATURE (11)..(11): Xaa is present or absent and when present denotes Lys
  MISC_FEATURE (12)..(12): Xaa is present or absent and when present denotes Glu
  MISC_FEATURE (13)..(15): Xaa is present or absent and when present denotes Ala
  MISC_FEATURE (16)..(16): Xaa is present or absent and when present denotes Lys
  MISC_FEATURE (17)..(17): Xaa is present or absent and when present denotes Glu
  MISC_FEATURE (18)..(20): Xaa is present or absent and when present denotes Ala
  MISC_FEATURE (21)..(21): Xaa is present or absent and when present denotes Lys
  MISC_FEATURE (22)..(22): Xaa is present or absent and when present denotes Glu
  MISC_FEATURE (23)..(25): Xaa is present or absent and when present denotes Ala
  MISC_FEATURE (26)..(26): Xaa is present or absent and when present denotes Lys
AEAAAKXXXXXXXXXXXXXXXXXXXXA

SEQ ID NO: 52 - protein, 5 aa, Artificial sequence
  Synthetic peptide
PAPAP

SEQ ID NO: 53 - protein, 10 aa, Artificial sequence
  Synthetic peptide
  MISC_FEATURE (1)..(1): Xaa denotes any amino acid residue
  MISC_FEATURE (3)..(3): Xaa is present or absent and when present denotes any amino acid residue
  MISC_FEATURE (4)..(4): Xaa is present or absent and when present denotes Pro
  MISC_FEATURE (5)..(5): Xaa is present or absent and when present denotes any amino acid residue
  MISC_FEATURE (6)..(6): Xaa is present or absent and when present denotes Pro
  MISC_FEATURE (7)..(7): Xaa is present or absent and when present denotes any amino acid residue
  MISC_FEATURE (8)..(8): Xaa is present or absent and when present denotes Pro
  MISC_FEATURE (9)..(9): Xaa is present or absent and when present denotes any amino acid residue.
  MISC_FEATURE (10)..(10): Xaa is present or absent and when present denotes Pro
XPXXXXXXXX

SEQ ID NO: 54 - protein, 17 aa, Artificial sequence
  Synthetic peptide
VSQTSKLTRAETVFPDV

SEQ ID NO: 55 - protein, 6 aa, Artificial sequence
  Synthetic peptide
PLGLWA

SEQ ID NO: 56 - protein, 6 aa, Artificial sequence
  Synthetic peptide
RVLAEA

SEQ ID NO: 57 - protein, 10 aa, Artificial sequence
  Synthetic peptide
EDVVCCSMSY

SEQ ID NO: 58 - protein, 8 aa, Artificial sequence
  Synthetic peptide
GGIEGRGS

SEQ ID NO: 59 - protein, 10 aa, Artificial sequence
  Synthetic peptide
TRHRQPRGWE

SEQ ID NO: 60 - protein, 10 aa, Artificial sequence
  Synthetic peptide
AGNRVRRSVG

SEQ ID NO: 61 - protein, 9 aa, Artificial sequence
  Synthetic peptide
RRRRRRRRR

SEQ ID NO: 62 - protein, 4 aa, Artificial sequence
  Synthetic peptide
GFLG

SEQ ID NO: 63 - protein, 12 aa, Artificial sequence
  Synthetic peptide
CRRRRRREAEAC

SEQ ID NO: 64 - protein, 26 aa, Artificial sequence
  Synthetic peptide
GYKRKFHEKHHSHRYGDYGSNYLYDN

SEQ ID NO: 65 - protein, 27 aa, Artificial sequence
  Synthetic peptide
HEKHHHEKHHHEKHHYGDYGSNYLYDN

SEQ ID NO: 66 - protein, 30 aa, Artificial sequence
  Synthetic peptide
HEKRHHHEKRHHHEKRHHYGDYGSNYLYDN

SEQ ID NO: 67 - protein, 29 aa, Artificial sequence
  Synthetic peptide
HEKRHHHEKRHHHEKHHYGDYGSNYLYDN

SEQ ID NO: 68 - protein, 28 aa, Artificial sequence
  Synthetic peptide
HEKRHHHEKHHHEKHHYGDYGSNYLYDN

SEQ ID NO: 69 - protein, 16 aa, Artificial sequence
  Synthetic peptide
RQIKIWFQNRRMKWKK

SEQ ID NO: 70 - protein, 40 aa, Artificial sequence
  Synthetic peptide
  MOD_RES (3)..(3): PHOSPHORYLATION
SDSHEKRHHGYRRKFHEKHHSHREFPFYGDYGSNYLYDNS

SEQ ID NO: 71 - protein, 38 aa, Artificial sequence
  Synthetic peptide
  MOD_RES (35)..(35): PHOSPHORYLATION
HYHKFHRYYDPGSNLYKEHNHGFHHGYKDEFRRESRDS

SEQ ID NO: 72 - protein, 69 aa, Artificial sequence
  Synthetic peptide
MTTLIPILSTFLFEDFSKASGFKGQRPETLHERLTLVSVYAPYLLIPFILLIFMLRSPYYKYEEKRKKK

SEQ ID NO: 73 - protein, 36 aa, Artificial sequence
  Synthetic peptide
MTTLIPILSTFLFEDFSKASGFKGQRPETLHERLTL

SEQ ID NO: 74 - protein, 33 aa, Artificial sequence
  Synthetic peptide
VSVYAPYLLIPFILLIFMLRSPYYKYEEKRKKK

SEQ ID NO: 75 - protein, 19 aa, Artificial sequence
  Synthetic peptide
DSHAKRHHGYKRKFHEKHH

(19) United States
(12) Patent Application Publication
(10) Pub. No.: US 2023/0190870 A1
(43) Pub. Date: Jun. 22, 2023

(54) Method for Treating Lysosomal Storage Diseases with Histatin Peptides

(71) Applicant: THE BOARD OF TRUSTEES OF THE UNIVERSITY OF ILLINOIS, Urbana, IL (US)

(72) Inventor: Vinay Kumar AAKALU, Chicago, IL (US)

(21) Appl. No.: 17/925,361

(22) PCT Filed: May 20, 2021
(86) PCT No.: PCT/US2021/033319

§ 371 Date: Nov. 15, 2022

Related U.S. Application Data
(60) Provisional application No. 63/027,885, filed on May 20, 2020.

(51) Int. Cl. A61K 38/17; A61P 3/06

(52) U.S. Cl. CPC A61K 38/1709; A61P 3/06

(57) ABSTRACT

Methods are provided for using histatins in the treatment of Neimann Pick Type C Disease and other diseases associated with TMEM97 (sigma 2 receptor) or NPC1 activity and/or cholesterol or microtubule-associated protein 1 light chain 3 accumulation. Methods for modulating TMEM97 and/or NPC1 activity in the treatment of ocular diseases is also provided.

### INTRODUCTION

This application claims benefit of priority to U.S. Provisional Patent Application Ser. No. 63/027,885, filed May 20, 2020, the content of which is incorporated herein by reference in its entirety.

This invention was made with government support under grant number EY024339, EY029409, EY001792, EY024710 and EY029426 awarded by the National Institutes of Health; and W81XWH-17-1-0122 awarded by the Department of Defense; and I01BX004080 awarded by the Department of Veterans Affairs Office of Research and Development. The government has certain rights in the invention.

### BACKGROUND

Histatins (HTNs) are small histidine-rich cationic peptides found in saliva, as well as human lacrimal epithelium (Aakalu, et al. (2014) Invest. Ophthalmol. Vis. Sci. 55:3115; Ubels, et al. (2012) Invest. Ophthalmol. Vis. Sci. 53(11):6738-47; Steele, et al. (2002) Invest. Ophthalmol. Vis. Sci. 43:98). Histatins range in size from 7 to 38 amino acid residues in length and represent a group of antimicrobial peptides with antibacterial properties and significant antifungal properties. In addition, histatins have been implicated in wound healing, metal ion chelation, anti-inflammatory effects and angiogenesis (Melino, et al. (2014) FEBS J. 281:657-72; Oudhoff, et al. (2008) FASEB J. 22(12):3805-12); Oudhoff, et al. (2009) J. Dent. Res. 88(9):846-50; WO 2007/142381). Structure-function studies have identified distinct N-terminal and C-terminal domains in both HTN1 and HTN3, which respectively contribute to the antimicrobial and wound healing properties (Melino, et al. (1999) Biochemistry 38:9626-33; Brewer, et al. (1998) Biochem. Cell Biol. 76:247-56; Gusman, et al. (2001) Biochim. Biophys. Acta 1545:86-95). In this respect, histatins, as well as fragments, multimers and combinations thereof, have been suggested for use in treating various conditions including ocular surface disease (US 2013/0310327; US 2013/0310326; US 2017/0239330; WO 2016/060916; WO 2016/060917; WO 2016/060918; WO 2016/060921; US 2016/0279194; WO 2017/095769) and wounds (US 2013/0288964; US 2011/0178010).

Cyclic analogs of histatins have also been described. For example, U.S. Pat. No. 6,555,650 describes cyclic analogues of HTN5 with disulfide bridges that create a cyclic portion of from 5-16 of said amino acid units. In addition, head-to-tail cyclization of HTN5 has been shown to increase amphipathicity of the peptide without affecting its antimicrobial potency (Sikorska & Kamysz (2014) J. Pept. Sci. 20:952-7). Further, cyclization of histatin-1 has been shown to potentiate the molar activity approximately 1000-fold (Oudhoff, et al. (2009) FASEB J. 23:3928-35) and increases wound closure activity (Bolscher, et al. (2011) FASEB J. 25:2650-8). Moreover, cyclic analogs of histatin, with enhanced potency have been suggested for use in treating microbial infection (US 2010/0173833; Brewer & Lajoie (2002) Biochemistry 41:5526-5536).

### SUMMARY OF THE INVENTION

This invention provides methods of treating a lysosomal storage disorder (e.g., a glycogen storage disease, mucopolysaccaridosis, mucolipidosis, oligosaccharidosis, sphingolipidosis, lysosomal transport disease or lipidosis such as Niemann-Pick type C disease) and reducing accumulation of cholesterol, modulating calcium signaling, decreasing apoptotic signaling, reducing losses in cell viability, or reducing microtubule-associated protein 1 light chain 3 protein accumulation by administering to a subject in need of treatment an effective amount of one or a combination of histatin peptides. In some aspects, the histatin peptide is a native histatin or synthetic histatin, e.g., a peptide that is linear or cyclized and optionally modified by glycosylation, acetylation, amidation, formylation, hydroxylation, methylation, myristoylation, phosphorylation, sulfonation, PEGylation or lipidation. When used in the methods of this invention, the histatin may be formulated for topical, oral, ocular, intravenous, intravitreal, subconjunctival, subcutaneous, intramuscular, intraperitoneal, intracerebral, intraarterial, intraportal, intralesional, intrathecal, or intranasal administration. Ideally, the histatin is formulated in the form of a gel, wash, cream, tablet, capsule, pill, solution, eye drop, spray, bandage, contact lens, depot, injectable, implantable, sustained-release or microparticle or nanoparticle formulation.

This invention also provides a method for treating an ocular disease or condition by administering to a subject in need of treatment and effective amount of a TMEM97 modulator to treat the ocular disease or condition. In some embodiments, the effective amount promotes wound healing and epithelial cell migration promoting activity in ocular tissue.

### BRIEF DESCRIPTION OF THE DRAWINGS

FIG. 1 shows that siRNA-mediated knockdown of TMEM97 inhibits Hst1 induced human corneal epithelial (HCE) migration. Shown are cell counts of transmigrated HCE cells in the Boyden chamber assay with and without siRNA knockdown of TMEM97 over a range of concentrations of Hst1. Statistical significance was determined by 2-way ANOVA with Bonferroni's post-hoc test. **p<0.01. Error bars indicate Standard Error of the Mean. Experiments were performed in triplicate. Statistical analyses were performed using GraphPad Prism software 5.0 (GraphPad Software, La Jolla, Calif.).

FIG. 2 shows that siRNA-mediated knockdown of TMEM97 inhibits Hst1 induced HCE wound closure. Shown is a bar graph depicting scratch closure % over time. Notably, a statistically significant improvement in scratch closure rates was found (versus untreated control) with Hst1 treatment (20 or 50 μM) of concentrations at 8 and 16 hours and loss of this response to Hst1 application in the TMEM97 KD cells. Statistical significance was determined by 1-way ANOVA with Bonferroni's post-hoc test. *p<0.05; **p<0.01. Error bars indicate Standard Error of the Mean. Experiments were performed in triplicate. Statistical analyses were performed using GraphPad Prism software 5.0 (GraphPad Software, La Jolla, Calif.).

FIG. 3 shows that treatment of human corneal epithelial cells with histatin peptides increases cellular calcium levels.

FIG. 4 shows that treatment of NPC1 (I1061T mutant) homozygous patient fibroblasts with histatin peptides increases cellular calcium levels.

FIG. 5 shows that human corneal epithelial cells treated with benzalkonium chloride (BAK) have induction of cell death/loss of cell viability, which is abrogated by treatment with histatin peptides.

FIG. 6 shows that human corneal epithelial cells treated with hyperosmolarity exhibit an induction of apoptotic signals, which is abrogated by treatment with histatin peptides.

### DETAILED DESCRIPTION OF THE INVENTION

It has now been demonstrated that histatin peptides antagonizes the binding of some ligands for TMEM97 and reduces findings of NPC disease. Interaction with the TMEM97 receptor has broad implications in a variety of disorders related to this receptor, and its primary interacting protein Niemann-Pick C1 (or NPC Intracellular Cholesterol Transporter 1; NPC1). There are a number of diseases and conditions associated with the activities of TMEM97 and NPC1 including inflammation, cancer and neurodegenerative disorders. Accordingly, this invention is the use of one or more agents (e.g., histatin peptides or TMEM97 modulators) to modulate (e.g., antagonize) the activity of TMEM97 and/or modulate NPC1 activity for treating a disease or condition associated with TMEM97 and/or NPC1 activity (e.g., Niemann-Pick type C disease or ocular diseases or conditions).

The Sigma-2 receptor (S2R) also known as the endoplasmic reticular protein Transmembrane Protein 97 (TMEM97), is a critical component of cholesterol processing in many cell types. It is an endoplasmic reticulum resident transmembrane protein that regulates the sterol transporter NPC1. TMEM97 is implicated in a number of diseases including cancer and neurodegenerative diseases. The TMEM97 protein was originally described pharmacologically, with disparate small molecules and drugs found to target this protein and exhibit efficacy in the treatment of cancer, pain, Alzheimer's disease, aging and mitochondrial disorders and multiple sclerosis. In addition, siRNA knockdowns of TMEM97 have been demonstrated to ameliorate some of the findings associated with NPC1 loss of function mutations.

NPC1 (and NPC2) is a transporter that is frequently mutated, causing loss or reduction of function, in the lysosomal storage disorder Neimann Pick Type C Disease. Without adequate NPC1 function, free cholesterol collects inside cells. The consequences of this defect are broad and severe. NPC1 is extremely important to many diseases including associations with Alzheimer's disease, Crohn's disease, abnormal platelet function and formation, movement disorders, neurologic dysfunction, liver and lung disease, susceptibility to infections like Ebola virus (including ocular conjunctivitis), chronic inflammation, defective bacterial and microbial killing, constitutive Toll-like receptor 4 activation, obesity, tuberous sclerosis, cerebrovascular disease, atherosclerosis amongst other diseases.

In accordance with this invention, histatins such as histatin 1, 3, 5 and other histatin peptides (e.g., native histatins 1, 2, 3, 4, 5, 6, 7, 8, 9, 10, 11, 12, 13 or synthetic variants thereof) can modulate the function of TMEM97 and the NPC1 pathway through pharmacological radioligand binding assays and immunoprecipitation and protein-protein interaction assays as well as functional assays. Therefore, this invention provides for the use of native histatins 1, 2, 3, 4, 5, 6, 7, 8, 9, 10, 11, 12, 13 and/or synthetic variants thereof in the methods of this invention. As used herein, “native histatin,” “endogenous histatin,” or “natural histatin” refers to a 7-44 amino acid residue, histidine-rich peptide, which was originally identified in saliva and characterized based upon its fungistatic effects. See, e.g., Melino, et al. (2014) FEBS J. 281:657-682, and references cited therein. Representative native histatins include the peptides listed in Table 1.

TABLE 1

| | | SEQ |
|---|---|---|
| | | ID |
| Histatin | Sequence | NO: |
| H1 | DSpHEKRHHGYRRKFHEKHHSHREFPFYGDYGS | 6 |
| | NYLYDN | |
| H2 | RKFHEKHHSHREFPFYGDYGSNYLYDN | 7 |
| H3 | DSHAKRHHGYKRKFHEKHHSHRGYRSNYLYDN | 8 |
| H4 | KFHEKHHSHRGYRSNYLYDN | 9 |
| H5 | DSHAKRHHGYKRKFHEKHHSHRGY | 10 |
| H6 | DSHAKRHHGYKRKFHEKHHSHRGYR | 11 |
| H7 | RKFHEKHHSHRGY | 12 |
| H8 | KFHEKHHSHRGY | 13 |
| H9 | RKFHEKHHSHRGYR | 14 |
| H10 | KFHEKHHSHRGYR | 15 |
| H11 | KRHHGYKR | 16 |
| H12 | KRHHGYK | 17 |

″Sp″ or ″S(PO₃)″ denotes phosphorylated serine.

In addition to native or natural histatin peptides, this invention also provides for the use of a synthetic histatin peptide, or a pharmaceutically acceptable salt thereof. A synthetic peptide of this invention has the general structure of Formula I:

Z—R¹—[L—R²]ₙ (I),

wherein

(i) at least one of R¹ or R² is a 5 to 10 amino acid residue peptide having the amino acid sequence or HEXXH (SEQ ID NO:1), wherein each X is independently R, K, or H; and the other of R¹ or R² is a metal binding peptide, wound healing peptide, or antimicrobial peptide;

(ii) Z is present or absent and when present is an exogenous peptide;

(iii) L is a linker, which may be present or absent; and

(iv) n is 0 or ≥1 with the proviso that when n is 0, R¹ is a 5 to 10 amino acid residue peptide having the amino acid sequence HEXXH (SEQ ID NO:1).

Ideally, at least one of R¹ and R² is a 5 to 10 amino acid residue peptide that includes the amino acid sequence HEXXH (SEQ ID NO:1), wherein each X is independently R (Arg), K (Lys), or H (His). Accordingly, at least one of R¹ and R² may be a 5, 6, 7, 8, 9 or 10 amino acid residue peptide that includes the amino acid sequence HEKKH (SEQ ID NO:18), HEKRH (SEQ ID NO:19), HEKHH (SEQ ID NO:20), HERKH (SEQ ID NO:21), HERRH (SEQ ID NO:22), HERHH (SEQ ID NO:23), HEHKH (SEQ ID NO:24), HEHRH (SEQ ID NO:25) or HEHHH (SEQ ID NO:26). At least one of R¹ or R² may include the sequence HEXXH (SEQ ID NO:1), which may have 1 to 5 additional amino acid residues on the C-terminus and/or N-terminus. In some aspects, the 1 to 5 additional amino acid residues are endogenous or native amino acid residues. A “native” or “endogenous” amino acid residue is an amino acid residue that is present at the recited position in a naturally occurring protein. By way of illustration, the sequence HEKHH (SEQ ID NO:20) is present within histatin 3 as follows: DSHAKRHHGYKRKFHEKHHSHRGYRSNYLYDN (SEQ ID NO:8). Accordingly, when R¹ and/or R² is derived from a histatin, R¹ and/or R² can have the sequence GYKRKFHEKHHSHR (SEQ ID NO:27), KRKFHEKHHSHR (SEQ ID NO:28), HEKHHSHR (SEQ ID NO:29) or HEKRHH (SEQ ID NO:30).

In some aspects, the synthetic peptide consists only of R¹ (i.e., n=0). In accordance with this aspect, the synthetic peptide is a 5, 6, 7, 8, 9 or 10 amino acid residue peptide comprising or consisting of the sequence set forth in SEQ ID NO:18, SEQ ID NO:19, SEQ ID NO:20, SEQ ID NO:21, SEQ ID NO:22, SEQ ID NO:23, SEQ ID NO:24, SEQ ID NO:25, or SEQ ID NO:26.

In other aspects, the synthetic peptide includes one or more R² peptides (i.e., n≥1). In this respect, the synthetic peptide can include 2, 3, 4, 5, 6, 7, 8, 9, 10, 11, 12, 13, 14, 15, 16, 17, 18, 19, 20 or more peptides joined by linkers. In one aspect R¹ and R² of the synthetic peptide of this invention are the same. In other aspects, R¹ and R² of the synthetic peptide of this invention are different. In further aspects, each R² can be the same or different. Ideally, the total length the synthetic peptide is in the range of 20 to 100 amino acid residues.

While at least one of R¹ or R² is a 5 to 10 amino acid residue peptide having the amino acid sequence HEXXH (SEQ ID NO:1), the other of R¹ or R² may be a metal binding peptide, wound healing peptide, or antimicrobial peptide. In this respect, a synthetic peptide of the invention may be composed of a 5 to 10 amino acid residue peptide having the amino acid HEXXH (SEQ ID NO:1) in combination with (i) a metal binding peptide, (ii) a wound healing peptide, (iii) an antimicrobial peptide, or (iv) any combination of (i)-(iii).

The term “metal binding peptide,” as used herein, refers to an amino acid motif that binds or forms a complex with a metal. Structural and functional characterization of histatins has revealed the presence of two metal-binding motifs: the amino-terminal Cu (II)/Ni (II) binding (ATCUN) motif with one histidine residue in the third position (NH₂—X¹X²H, wherein X¹ is Asp or Glu, and X² is Ala, Thr, Met or Ser) (Grogan, et al. (2001) FEBS Lett. 491:76-80; Melino, et al. (2006) Biochemistry 45:15373-83; Melino, et al. (1999) Biochemistry 38:9626-33; Gusman, et al. (2001) Biochim. Biophys. Acta 1545:86-95); and the Zn(II)-binding motif HEXXH (SEQ ID NO:1), wherein each X independently denotes a basic amino acid residue such as K (Lys), R (Arg), or H (His). Accordingly, in some embodiments, the metal binding peptide includes the sequence DSH, ESH, DAH, EAH, DTH, ETH, DMH or EMH. In other embodiments, the metal binding peptide includes the sequence HEKKH (SEQ ID NO:18), HEKRH (SEQ ID NO:19), HEKHH (SEQ ID NO:20), HERKH (SEQ ID NO:21), HERRH (SEQ ID NO:22), HERHH (SEQ ID NO:23), HEHKH (SEQ ID NO:24), HEHRH (SEQ ID NO:25) or HEHHH (SEQ ID NO:26). The metal binding peptide can include the specific sequence of the above-referenced metal binding peptides or can include between 1 and 6 additional native histatin amino acid residues on the C- and/or N-terminus of the metal binding peptide. By way of illustration, a metal binding peptide can have the sequence GYKRKFHEKHHSHR (SEQ ID NO:27), KRKFHEKHHSHR (SEQ ID NO:28), HEKHHSHR (SEQ ID NO:29) or HEKRHH (SEQ ID NO:29).

In some embodiments, a synthetic peptide of the invention includes one metal binding peptides. In other embodiments, a synthetic peptide includes two metal binding peptides. In further embodiments, a synthetic peptide includes three metal binding peptides. In certain embodiments, a metal binding peptide has the sequence HEXXH (SEQ ID NO:1), wherein each X independently denotes a basic amino acid residue. As would be readily appreciated by those of skill in the art, the inclusion of one or more metal binding peptides in a synthetic peptide impart metal ion chelating, anti-inflammatory, matrix metalloproteinase inhibitory, and/or anti-angiogenic activity to the synthetic peptide. In light of its anti-angiogenic activity, such a synthetic peptide would be of use in treating age-related macular degeneration, diabetic retinopathy, cancer, and chronic or acute sever uveitis. In light of its metal ion chelating activity, such a synthetic peptide would also be of use in inhibiting tissue destruction mediated by matrix metalloproteinases and other metal-dependent enzymes in inflammatory and infectious diseases such as infectious keratitis, intraocular uveitis, endophthalmitis, inflammatory keratitis, dry eye disease and ocular surface or intraocular diseases.

As used herein, “wound healing peptide” refers to an amino acid motif that promotes or facilitates wound healing. In some aspects, a wound healing peptide is derived from histatin. An example of a wound healing peptide derived from histatin is a peptide including the sequence SNYLYDN (SEQ ID NO:2). In another aspect, the wound healing peptide includes the amino acid sequence SHXGY (SEQ ID NO:3), wherein X is R, K, H, D or E. Accordingly, a wound healing peptide can have the amino acid sequence SHRGY (SEQ ID NO:31), SHDGY (SEQ ID NO:32), SHKGY (SEQ ID NO:33), SHHGY (SEQ ID NO:34), or SHEGY (SEQ ID NO:35). In some aspects, the wound healing peptide may have 1 to 5 additional amino acid residues on the C-terminus and/or N-terminus. For example, the 1 to 5 additional amino acid residues are native amino acid residues. By way of illustration, the sequence SHRGY (SEQ ID NO:31) is present within histatin 3 as follows: DSHAKRHHGYKRKFHEKHHSHRGYRSNYLYDN (SEQ ID NO: 8). Accordingly, when the wound healing peptide is derived from a histatin, said peptide can have the sequence HHSHRGYRSN (SEQ ID NO:36), HEKHHSHRGY (SEQ ID NO:37), EKHHSHRGYR (SEQ ID NO:38), KHHSHRGY (SEQ ID NO:39), HHSHRGY (SEQ ID NO:40), or HSHRGY (SEQ ID NO:41).

Notably, when included in the synthetic peptide of this invention, the SNYLYDN (SEQ ID NO:2) or SHXGY (SEQ ID NO:3) sequence has the additional advantage of conferring immunomodulatory activity to the synthetic peptide. The wound healing peptide can include the specific sequence of the above-referenced wound healing peptides or can include between 1 and 6 additional amino acid residues on the C- and/or N-terminus of the wound healing peptide. By way of illustration, a wound healing peptide derived from histatin can have the sequence YGDYGSNYLYDN (SEQ ID NO:42).

In some embodiments, in addition to the wound healing peptide of SEQ ID NO:2 or 3, the synthetic peptide of the invention includes a second wound healing peptide. In other embodiments, in addition to the wound healing peptide of SEQ ID NO:2 or 3, a synthetic peptide includes two additional wound healing peptides. In further embodiments, in addition to the wound healing peptide of SEQ ID NO:2 or 3, a synthetic peptide includes three additional wound healing peptides. As would be readily appreciated by those of skill in the art, the inclusion of one or more wound healing peptides in a synthetic peptide impart epithelial cell migration and spreading activity to the synthetic peptide. Such a synthetic peptide would therefore be of use in wound healing as well as the treatment of retinal pigment epithelial healing, dry age-related macular degeneration, ocular surface diseases and ocular surface inflammatory disorders, ocular neovascularization including corneal and intraocular, retinal or choroidal, and dry eye diseases.

For the purposes of this invention, “antimicrobial” includes both antibacterial and antifungal agents. Accordingly, the term “antimicrobial” peptide,” as used herein, refers to an amino acid motif that exhibits cytostatic or cytocidal activity toward bacterial and/or fungal cells. Characterization of histatins indicates that a positive net charge and the amino-terminal portion of HTNs mediate antimicrobial activity. In particular, the amino acid sequence RKFHEKHHSHRGYR (SEQ ID NO:4) of histatin 3 has been shown to exhibit fungicidal activity (Oppenheim, et al. (2012) PLoS ONE 7(12):e51479). Similarly, the sequence AKRHHGYKRKFH (SEQ ID NO:5), also known as P-113, exhibits fungicidal activity against Candida albicans (Jang, et al. (2008) Antimicrob. Agents Chemother. 5292):497-504). Thus, the antimicrobial peptide can include the specific sequence of the above-referenced antimicrobial peptides or can include between 1 and 6 additional amino acid residues on the C- and/or N-terminus of the antimicrobial peptide.

In some embodiments, a synthetic peptide includes one antimicrobial peptide. In other embodiments, a synthetic peptide includes two antimicrobial peptides. In further embodiments, a synthetic peptide includes three antimicrobial peptides. In certain embodiments, an antimicrobial peptide has the sequence RKFHEKHHSHRGYR (SEQ ID NO:4). In other embodiments, an antimicrobial domain has the sequence AKRHHGYKRKFH (SEQ ID NO:5). As would be readily appreciated by those of skill in the art, the inclusion of one or more antimicrobial peptides in a synthetic peptide impart antifungal and/or antibacterial activity to the synthetic peptide. Such a synthetic peptide would therefore be of use in treating microbial infections such as Candida eye infection as well as preventing infections associated with surgical implants.

Examples of synthetic peptides containing repeating units that are the same or different are presented in Table 2.

TABLE 2
Synthetic Peptide

HEKHH (SEQ ID NO: 20) -L-SHRGY (SEQ ID NO: 31)
HEKHH (SEQ ID NO: 20) -L-HEKHH (SEQ ID NO: 20) -L-SHRGY (SEQ ID
NO: 31) -L-YGDYGSNYLYDN (SEQ ID NO: 42)
SHRGY (SEQ ID NO: 31) -L-HEKRHH (SEQ ID NO: 30) -L-HEKRHH (SEQ ID
NO: 30) -L-YGDYGSNYLYDN (SEQ ID NO: 42)
GYKRKFHEKHHSHR (SEQ ID NO: 27) -L-YGDYGSNYLYDN (SEQ ID NO: 25)
HEKHH (SEQ ID NO: 20) -L-HEKHH (SEQ ID NO: 20) -L-HEKHH (SEQ ID
NO: 20) -L-YGDYGSNYLYDN (SEQ ID NO: 42)
HEKRHH (SEQ ID NO: 30) -L-HEKRHH (SEQ ID NO: 30) -L-HEKRHH (SEQ ID
NO: 30) -L-YGDYGSNYLYDN (SEQ ID NO: 42)
HEKRHH (SEQ ID NO: 30) -L-HEKRHH (SEQ ID NO: 30) -L-HEKHH (SEQ ID
NO: 20) -L-YGDYGSNYLYDN (SEQ ID NO: 42)
HEKRHH (SEQ ID NO: 30) -L-HEKHH (SEQ ID NO: 20) -L-HEKHH (SEQ ID
NO: 20) -L-YGDYGSNYLYDN (SEQ ID NO: 42)

In certain aspects of this invention, exogenous or heterologous molecules are included in the synthetic peptide. Specifically, in some aspects, the synthetic peptide optionally includes “Z” and/or “L” moieties directly attached to one or both of R¹ and R², wherein both “Z” and “L” moieties are exogenous or heterologous molecules with respect to R¹ and R². The term “heterologous molecule” or “exogenous molecule” refers to a molecule that is not normally found in a peptide or not typically associated with R¹ and/or R² amino acid sequences in nature.

In some aspects, the synthetic peptide includes a “Z” moiety. In other aspects, the “Z” moiety is absent. When present, Z is an exogenous peptide as defined herein. In accordance with this aspect, Z is a 1 to 50 amino acid residue peptide, or preferably a 1 to 30 amino acid residue peptide, or more preferably a 1 to 20 amino acid residue peptide, wherein said exogenous peptide may or may not have a function.

As used herein, the terms “L” or “linker” or “spacer” refers to a heterologous or exogenous molecule used to connect, link or join R¹ to R² and connect, link or join individual R² moieties. As used herein, the term “linked,” “joined” or “connected” generally refers to a functional linkage between two contiguous or adjacent amino acid sequences to produce a molecule that does not exist in nature. Generally, the linked amino acid sequences are contiguous or adjacent to one another and retain their respective operability and function when joined. The linkers may provide desirable flexibility to permit the desired expression, activity and/or conformational positioning of the synthetic peptide.

In some embodiments, a synthetic peptide includes one linker, i.e., n=1. In other embodiments, a synthetic peptide includes 2, 3, 4, 5, 6, 7, 8, 9, 10, 11, 12, 13, 14, 15, 16, 17, 18, or 19 linkers, i.e., n=2, 3, 4, 5, 6, 7, 8, 9, 10, 11, 12, 13, 14, 15, 16, 17, 18, or 19. In certain aspects, each occurrence of a linker (L) may include the same or different linker.

Linkers of use in the synthetic peptide of Formula I can be flexible, rigid, in vivo cleavable, or a combination thereof. In addition, linkers can be composed of amino acid residues (i.e., peptide linkers) or composed of chains of hydrocarbons (i.e., hydrocarbon linkers). Peptide linkers can be of any appropriate length to connect R¹ and R² or individual R² moieties and are preferably designed so as to allow the proper folding and/or function and/or activity of R¹ and R². Thus, the linker peptide can have a length of no more than 3, no more than 5, no more than 10, no more than 15, no more than 20, no more than 25, no more than 30, no more than 35, no more than 40, no more than 45, no more than 50, no more than 55, or no more than 60 amino acids. In some embodiments, the linker peptide can have a length of at least 3, at least 4, at least 5, at least 6, at least 7, at least 8, at least 9, at least 10, at least 12, at least 15, at least 18, at least 20, at least 25, at least 30, at least 35, at least 40, at least 45, or at least 50 amino acids. In some embodiments, the linker includes at least 10 and no more than 60 amino acids, at least 10 and no more than 55 amino acids, at least 10 and no more than 50 amino acids, at least 10 and no more than 45 amino acids, at least 10 and no more than 40 amino acids, at least 10 and no more 35 amino acids, at least 10 and no more than 30 amino acids, at least 10 and no more than 25 amino acids, at least 10 and no more than 20 amino acids or at least 10 and no more than 15 amino acids.

A “flexible” linker refers to a hydrocarbon or peptide linker that does not have a fixed structure (secondary or tertiary structure) in solution. Such a flexible linker is therefore free to adopt a variety of conformations. Flexible linkers of use herein include hydrocarbon linkers and peptide linkers composed of small, non-polar. (e.g., Gly) and/or polar (e.g., Ser or Thr) amino acid residues. Simple amino acids (e.g., amino acids with simple side chains (e.g., H, CH₃ or CH₂OH) are advantageous for use in a peptide linker as the lack of branched side chains on these amino acids provides greater flexibility (e.g., two-dimensional or three-dimensional flexibility) within the linker and, accordingly, within a polypeptide composition. The flexible linker may contain additional amino acids such as Thr and Ala to maintain flexibility, as well as polar amino acids such as Lys and Glu to improve solubility. The amino acids can alternate/repeat in any manner consistent with the linker remaining functional (e.g., resulting in expressed and/or active polypeptide(s)). Flexible linkers are described, for example, in Chen, et al. (2013) Adv. Drug Deliv. Rev. 65(10):1357-1369; US 2012/0232021; US 2014/0079701; WO 1999/045132; WO 1994/012520 and WO 2001/1053480.

In particular aspects, the flexible linker is a hydrocarbon linker. The hydrocarbon linking R¹ and R² or individual R² moieties should have sufficient length and flexibility so that the synthetic peptide can achieve the desired conformation. In certain embodiments, the hydrocarbon is composed of one or more methylene (—CH₂—) groups. In certain embodiments, the hydrocarbon includes between 3 and 25 methylene groups, i.e., —(CH₂)ₙ—, wherein n is 3 to 25. In certain embodiments, the hydrocarbon linker has the structure —(CH₂)₆—. Additional carbon-based linkers such as glycol linkers could also be used in the synthetic peptide of this invention.

In other embodiments, the linker is a rigid linker. “Rigid” linker refers to a molecule that adopts a relatively well-defined conformation when in solution. Rigid linkers are therefore those which have a particular secondary and/or tertiary structure in solution. Rigid linkers are typically of a size sufficient to confer secondary or tertiary structure to the linker. Such linkers include aromatic molecules (see, e.g., U.S. Pat. No. 6,096,875 or U.S. Pat. No. 5,948,648), peptide linkers rich in proline, or peptide linkers having an inflexible helical structure. Rigid linkers are described in, for example, Chen, et al. (2013) Adv. Drug Deliv. Rev. 65(10):1357-1369; US 2010/0158823 and US 2009/10221477.

In other embodiments, the linker is an in vivo cleavable linker. In vivo cleavable linkers can include a cleavable disulfide bond formed between two cysteine residues or linkers having a protease recognition sequence, e.g., recognized by matrix metalloproteases (MMPs).

Examples of suitable peptide linkers of use in the synthetic peptide are provided in Table 3.

TABLE 3

| Type | Sequence | SEQ ID NO: |
|---|---|---|
| Flexible | (GGGGS)ₙ | 43 |
| Flexible | KESGSVSSEQLAQFRSLD | 44 |
| Flexible | EGKSSGSGSESKST | 45 |
| Flexible | GGGGGGGG | 46 |
| Flexible | GSAGSAAGSGEE | 47 |
| Flexible | (GGSG)ₙ | 48 |
| Flexible | (GS)ₙ | 49 |
| Rigid | (EAAAK)ₙ | 50 |
| Rigid | A(EAAAK)ₙA | 51 |
| Rigid | PAPAP | 52 |
| Rigid | (XP)ₙ | 53 |
| Cleavable | VSQTSKLTRAETVFPDV | 54 |
| Cleavable | PLGLWA | 55 |
| Cleavable | RVLAEA | 56 |
| Cleavable | EDVVCCSMSY | 57 |
| Cleavable | GGIEGRGS | 58 |
| Cleavable | TRHRQPRGWE | 59 |
| Cleavable | AGNRVRRSVG | 60 |
| Cleavable | RRRRRRRRR | 61 |
| Cleavable | GFLG | 62 |
| Cleavable | CRRRRRREAEAC | 63 |

n is 1 to 5. X may be any amino acid residue, but is preferably Ala, Lys or Glu.

Each of the individual linkers of the synthetic peptide of this invention can be the same or different. In some embodiments, a synthetic peptide includes at least one flexible linker. In some embodiments, at least one flexible linker is a hydrocarbon linker. In other embodiments, at least one flexible linker is a peptide linker. In particular embodiments, each linker of the synthetic peptide is a hydrocarbon linker. In certain embodiments, each linker of the synthetic peptide has the structure —(CH₂)₆—.

Examples of synthetic peptides containing combinations repeating units with flexible linkers are presented in Table 4.

TABLE 4

| Synthetic Histatin | SEQ ID NO: |
|---|---|
| GYKRKFHEKHHSHR-(CH₂)₆-YGDYGSNYLYDN | 64 |
| HEKHH-(CH₂)₆-HEKHH-(CH₂)₆-HEKHH-(CH₂)₆- | 65 |
| YGDYGSNYLYDN | |
| HEKRHH-(CH₂)₆-HEKRHH-(CH₂)₆-HEKRHH-(CH₂)₆- | 66 |
| YGDYGSNYLYDN | |
| HEKRHH-(CH₂)₆-HEKRHH-(CH₂)₆-HEKHH-(CH₂)₆- | 67 |
| YGDYGSNYLYDN | |
| HEKRHH-(CH₂)₆-HEKHH-(CH₂)₆-HEKHH-(CH₂)₆- | 68 |
| YGDYGSNYLYDN | |

In some aspects, a native or synthetic histatin peptide of the invention is prepared as a pharmaceutically acceptable salt. As used herein, the term “pharmaceutically acceptable salt” refers to those salts of the synthetic peptide which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well-known in the art. See, e.g., Berge, et al. (1977) J. Pharmaceutical Sciences 66:1-19. Salts can be prepared in situ during the final isolation and purification of the peptides of the invention, or separately by reacting a free base with a suitable organic acid. Examples of pharmaceutically acceptable salts include, but are not limited to, nontoxic acid addition salts formed from amino group and an inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

The native or synthetic histatin peptides described herein can be synthesized by routine methods including recombinant protein expression, chemical synthesis, or a combination thereof. In some embodiments, the peptide of the invention is synthesized recombinantly using recombinant DNA techniques. Thus, the invention provides polynucleotides that encode such peptides. In a related aspect, the invention provides vectors, particularly expression vectors that harbor the polynucleotides encoding the peptides of the invention. In certain embodiments, the vector provides replication, transcription and/or translation regulatory sequences that facilitate recombinant synthesis of the desired peptide in a eukaryotic cell or prokaryotic cell. Accordingly, the invention also provides host cells for recombinant expression of the peptide and methods of harvesting and purifying the synthetic peptide produced by the host cells. Production and purification of recombinant peptides is a routine practice to one of skilled in the art and any suitable methodology can be used.

In another embodiment, the native or synthetic histatin is synthesized by any of the chemical synthesis techniques known in the art, particularly solid-phase synthesis techniques, for example, using commercially-available automated peptide synthesizers. See, for example, Stewart & Young (1984) Solid Phase Peptide Synthesis, 2^{nd}ed., Pierce Chemical Co.; Tarn, et al. (1983) J. Am. Chem. Soc. 105:6442-55; Merrifield (1986) Science 232:341-347; and Barany et al. (1987) Int. J. Peptide Protein Res. 30:705-739.

The native or synthetic histatin can be isolated and/or purified by any suitable methods known in the art including without limitation gel filtration and affinity purification. In some embodiments, the peptide is produced with a tag, e.g., an epitope tag, to facilitate isolation of the peptide. In one aspect, the peptide is at least 1% pure, e.g., at least 5% pure, at least 10% pure, at least 20% pure, at least 40% pure, at least 60% pure, at least 80% pure, and at least 90% pure, as determined by SDS-PAGE. Once isolated and/or purified, the properties of the peptide can be readily verified by techniques known to those skilled in the art.

Derivatives and analogs of the peptides described herein are all contemplated and can be made by altering their amino acid sequences by substitutions, additions, and/or deletions/truncations or by introducing chemical modifications that result in functionally equivalent molecules. It will be understood by one of ordinary skill in the art that certain amino acids in a sequence of any polypeptide may be substituted for other amino acids without adversely affecting the activity of the polypeptides.

In certain embodiments, the native or synthetic histatin peptide of the invention includes one or more modifications including without limitation phosphorylation, glycosylation, hydroxylation, sulfonation, amidation, acetylation, carboxylation, palmitylation, PEGylation, introduction of nonhydrolyzable bonds, and disulfide formation. The modification may improve the stability and/or activity of the peptide.

For example, the C-terminal may be modified with amidation, addition of peptide alcohols and aldehydes, addition of esters, or addition of p-nitroaniline and thioesters. The N-terminal and side chains may be modified by PEGylation, acetylation, formylation, addition of a fatty acid, addition of benzoyl, addition of bromoacetyl, addition of pyroglutamyl, succinylation, addition of tetrabutyoxycarbonyl and addition of 3-mercaptopropyl, acylations (e.g., lipopeptides), biotinylation, phosphorylation, sulfation, glycosylation, introduction of maleimido group, chelating moieties, chromophores or fluorophores.

In one embodiment, the native or synthetic histatin peptide is conjugated to a fatty acid, e.g., the peptide is myristylated. For example, a fatty acid may be conjugated to the N-terminus of the peptide. Such fatty acids include caprylic acid, capric acid, lauric acid, myristic acid, palmitic acid, stearic acid, etc. Furthermore, cysteines in the peptide can be palmitoylated. In one embodiment, the peptide is myristylated, stearylated or palmitoylated at the N-terminal amino acid.

In addition, or as an alternative, to post-translational modifications, the peptide can be conjugated or linked to another peptide, such as a carrier peptide. The carrier peptide may facilitate cell-penetration and can include peptides such as antennapedia peptide, penetratin peptide, TAT, transportan or polyarginine. In an embodiment, the native or synthetic histatin peptide is conjugated or linked to the antennapedia peptide, RQIKIWFQNRRMKWKK (SEQ ID NO:69).

A native or synthetic histatin peptide of the invention may also be cyclized. As used herein the term “cyclized” or “cyclic” denote an analog of a linear peptide that incorporates at least one bridging group (e.g., an amide, thioether, thioester, disulfide, urea, carbamate, hydrocarbon or sulfonamide) between to amino acid residues to form a cyclic structure. The bridging group can present on the side chain of an amino acid residue or a terminal amino acid residue thereby providing side chain cyclization (e.g., lactam bridge, thioester), head-to-tail cyclization, or hydrocarbon-stapled peptides.

In certain embodiments, the cyclic peptide has a disulfide bridge between two terminal cysteine residues. A representative cyclized synthetic peptide is provided in Table 5.

TABLE 5

| | SEQ ID |
|---|---|
| Cyclic Synthetic Peptide | NO: |
| S-DSpHEKRHHGYRRKFHEKHHSHREFPFYGDYGSNYL | 70 |
| YDN-S | |

″Sp″ or ″S(PO₃)″ denotes phosphorylated serine.

In other embodiments, the cyclic peptide is prepared from a linear peptide by cyclization with sortase. “Cyclization with sortase” or “cyclized with sortase” refers to a method of cyclizing a linear peptide using the enzyme sortase. Sortase-based cyclization is known in the art for manufacturing large cyclic peptides. See, Bolscher, et al. (2011) FASEB J. 25(8):2650-2658, and references cited therein.

Butelase cyclization has also been used to cyclize peptides. Addition of the tripeptide Asn-His-Val motif at the C-terminus provides a substrate for butelase to cyclize a synthetic peptide at a rate significantly faster than that of sortase A. See, Nguyen, et al. (2016) Nat. Protocols 11:1977-88; Tam, et al. (June 2015) Peptides 2015: Proc. 24^{th} Am. Pept. Symp., Orlando, Fla., pg. 27.

In accordance with the principles herein, histatin peptides can modulate cholesterol localization, modulate calcium signaling, modulate apoptotic signaling, and modulate cell death and reduce the phenotype of NPC1 deficiency, and thereby affect the treatment of a number of diseases and disease phenotypes including, but not limited to, Niemann-Pick Type C disease, neurodegeneration (e.g., Alzheimer's disease), traumatic brain injury, chronic pain, cancer, obesity, insulin resistance, metabolic syndrome, hypercholesterolemia, liver disease, fatty liver metabolic disorders, steatosis, non-alcoholic steatohepatitis, hepatosplenomegaly, type 2 diabetes, weight gain, dyslipidemia, and the like. One method by which this result is demonstrated is in standardized phenotypic assays using Filipin staining, caspase assays, calcium staining, and cell viability assays. The results presented herein demonstrate the ability of histatins to reduce the accumulation of cholesterol, decrease calcium levels in wild-type cells, increase calcium levels in NPC1 cells, deduces losses in cell viability due to toxic assaults, and reduce LC3 accumulation in NPC1 models. Therefore, this invention also provides for methods to reduce the accumulation of cholesterol, modulate calcium signaling, decrease apoptotic signaling, reduce losses in cell viability, reduce LC3 protein accumulation, and in the treatment of NPC1 disease, NPC2 disease, as well as other lysosomal storage diseases.

Various lysosomal storage diseases, which may be classified in various ways, are within the scope of the present disclosure. In one embodiment, the lysosomal storage disease is chosen from any of glycogen storage disease, mucopolysaccaridoses, mucolipidoses, oligosaccharidoses, lipidoses, sphingolipidoses, and lysosomal transport diseases. The sphingolipidoses may be chosen from any of Niemann-Pick disease type A/B, Gaucher disease type I/II/III, Krabbe disease, Fabry disease, Schindler Disease, GM1 gangliosidosis, Morquio B disease, GM2 gangliosidoses, metachromatic leukodystrophy, Farber disease, multiple sulfatase deficiency, lysosomal acid lipase deficiency, galactosialidosis, Tay-Sachs disease, the AB variant of Tay-Sachs disease, and Sandhoff disease. The mucolipidoses may be chosen from any of mucolipidosis I, mucolipidosis II, mucolipidosis III, and mucolipidosis IV. The oligosaccharidoses may be chosen from any of beta-mannosidosis, alpha-fucosidosis, and aspartylglucosaminuria. In accordance with another aspect, the oligosaccharidosis is aspartylglucosaminuria. The lipidoses may be chosen from any of Niemann-Pick disease type C, Niemann-Pick disease type D, neuronal ceroid lipofuscinoses (Type I to X inclusive), and Wolman disease. In one embodiment, the lipidosis is Niemann-Pick disease type C.

The glycogen storage disease may be chosen from Infantile-onset Pompe disease, Late-onset Pompe disease and Danon disease. The lysosomal transport diseases may be chosen from cystinosis, pycnodysostosis, sialic acid storage disease and infantile free sialic acid storage disease.

The lysosomal storage disease may be a primary lysosomal hydrolase defect, a post-translational processing defect of lysosomal enzymes, a trafficking defect for lysosomal enzymes, a defect in lysosomal enzyme protection, a defect in soluble non-enzymatic lysosomal proteins, a transmembrane (non-enzyme) protein defect or an unclassified defect.

In one embodiment, the lysosomal storage disease is chosen from a primary lysosomal hydrolase defect. Primary lysosomal hydrolase defects include, but are not limited to, Tay-Sachs disease (β-hexosaminidase A defect), Sandhoff disease (β-hexosaminidase A+B defect), Fabry disease (α-galactosidase A defect), Krabbe disease (β-galactosyl ceramidase defect), Niemann-Pick Type A and B (sphingomyelinase defect), metachromatic leukodystrophy (arylsulphatase A defect), MPS IH (Hurler syndrome; α-iduronidase defect), MPS IS (Scheie syndrome; α-iduronidase defect), MPS IH-S(Hurler-Scheie syndrome; α-iduronidase defect), MPS II (Hunter syndrome; iduronate sulphatase defect), MPS IIIA (Sanfilippo A syndrome; heparan sulphamidase defect), MPS IIIB (Sanfilippo B syndrome; acetyl α-glucosaminidase defect), MPS IIIC (Sanfilippo C syndrome; acetyl CoA: α-glucosaminide N-acetyltransferase defect), MPS IIID (Sanfilippo D syndrome; N-acetyl glucosamine-6-sulphatase defect), MPS IV A (Morquio A disease; acetyl galactosamine-6-sulphatase defect), MPS IVB (Morquio B disease; β-galactosidase defect), MPS V (redesignated MPS IS), MPS VI (Maroteaux Lamy Syndrome; acetyl galactosamine-4-sulphatase (arylsulphatase B) defect), MPS VII (Sly Syndrome; β-glucuronidase defect), MPS IX (hyaluronidase defect), Wolman/cholesteryl ester storage disease (WD; acid lipase defect), Pompe disease (Type II; α 1,4-glucosidase defect), aspartylglucosaminuria (glycosylasparaginase defect), fucosidosis (α-fucosidase defect), α-mannosidosis (α-mannosidase defect), β-mannosidosis (p-mannosidase defect), Schindler disease (N-acetylgalactosaminidase defect), sialidosis/ML I (α-neuraminidase defect), infantile neuronal ceroid lipofuscinosis (CLN1; palmitoyl protein thioesterase defect), late infantile neuronal ceroid lipofuscinosis (CLN2; carboxypeptidase defect), early infantile GM1 gangliosidosis, late infantile GM1 gangliosidosis, adult infantile GM1 gangliosidosis, Gaucher Disease Type 1 (Non-Neuronopathic), Gaucher Disease Type 2/3 (Neuronopathic), Neuronal Ceroid Lipofuscinosis Type 4 (CLN4; Kufs disease; Adult NCL; palmotoyl-protein thioesterase-1 deficiency (Type A); Cathepsin F deficiency (Type B)), Neuronal Ceroid Lipofuscinosis Type 4 (CLN10; Congenital Cathepsin D Deficiency), Pycnodysostosis (Cathepsin K defect), Infantile-Onset Pompe Disease, Late-Onset Pompe Disease, Farber Disease (Farber's lipogranulomatosis; ceramidase deficiency; Fibrocytic dysmucopolysaccharidosis; Lipogranulomatosis) and Galactosialidosis (protective protein cathepsin A defect, PPCA defect). In one embodiment, the primary lysosomal hydrolase defect is chosen from Tay-Sachs disease, Sandhoff disease, Niemann-Pick Type A, Niemann-Pick Type B, neuronal ceroid lipofuscinoses, Gaucher disease, Fabry disease, Krabbe disease, GM1 gangliosidosis, GM2 gangliosidosis, metachromatic leukodystrophy, and Farber disease. In one embodiment, the primary lysosomal hydrolase defect is chosen from Tay-Sachs disease, Sandhoff disease, Niemann-Pick Type A, Niemann-Pick Type B, and GM1 gangliosidosis.

In one aspect, the lysosomal storage disease is chosen from a post-translational processing defect of lysosomal enzymes. Post-translational processing defects of lysosomal enzymes include, but are not limited to, mucosulphatidosis (MSD; multiple sulphatase defect), MLII (I-cell disease; N-acetyl glucosamine phosphoryl transferase defect) and MLIII (pseudo-Hurler polydystrophy; N-acetyl glucosamine phosphoryl transferase defect).

In another aspect, the lysosomal storage disease is chosen from a trafficking defect for lysosomal enzymes. Trafficking defects for lysosomal enzymes include, but are not limited to, mucolipidosis type II (I-cell disease; N-acetyl glucosamine phosphoryl transferase defect), mucolipidosis type IDA (pseudo-Hurler polydystrophy; N-acetyl glucosamine phosphoryl transferase defect) and mucolipidosis type IIIC.

In a further aspect, the lysosomal storage disease is a defect in lysosomal enzyme protection. Defects in lysosomal enzyme protection include, but are not limited to, galactosialidosis (protective protein cathepsin A (PPCA) defect).

In yet another aspect, the lysosomal storage disease is a defect in soluble non-enzymatic lysosomal proteins. Defects in soluble non-enzymatic lysosomal proteins include, but are not limited to, GM2 activator protein deficiency (variant AB), Niemann-Pick Disease Type C2 (NPC2), sphingolipid activator protein (SAP) deficiency.

In still a further aspect, the lysosomal storage disease is a transmembrane (non-enzyme) protein defect. Transmembrane (non-enzyme) protein defects include, but are not limited to, Danon disease (lysosome-associated membrane protein 2 (LAMP2) defect), NPC (NPC1 defect), cystinosis (cystinosin defect), infantile free sialic acid storage disease (ISSD; sialin defect), Salla disease (free sialic acid storage; sialin defect), juvenile neuronal ceroid lipofuscinosis (CLN3, Batten disease), adult neuronal ceroid lipofuscinosis (Kufs disease; Adult NCL; palmotoyl-protein thioesterase-1 deficiency (Type A); Cathepsin F deficiency (Type B)), neuronal ceroid lipofuscinoses (NCL) (CLN6, CLN7, and CLN8) and mucolipidosis type IV (mucolipin defect).

In a particular aspect, the lysosomal storage disease is Niemann-Pick Type C1 or Niemann-Pick Type C2. Niemann-Pick diseases are a heterogeneous group of autosomal recessive lysosomal storage diseases. Common cellular features include abnormal sphingomyelin (SM) storage in mononuclear phagocytic cells and parenchymal tissues, as well as (hepato)splenomegaly. Among the three main subgroups (A-C), NPC is classified as a fatal neurovisceral lysosomal storage disease caused by abnormal intracellular cholesterol transport-induced accumulation of unesterified cholesterol in late endosome/lysosomal compartments. Outside the CNS, the cellular characteristics of NPC include abnormal accumulation of unesterified cholesterol and other lipids (e.g., GSLs) within late endosome/lysosomal compartments. Conversely, there is no net elevation in cholesterol in the CNS (although it does have an altered distribution) but there are highly elevated levels of GSLs. Progressive neurodegeneration is particularly characterized by sequential degeneration of GABAergic Purkinje neurons in the cerebellum, which parallels the onset and progression of cerebellar ataxia and other aspects of neurological dysfunctions seen during the course of NPC. Genetic studies have shown that NPC disease is caused by mutations in either the Npc1 or Npc2 genes. NPC1 encodes a multimembrane spanning protein of the limiting membrane of the late endosome/lysosome, whereas NPC2 is a soluble cholesterol binding protein of the lysosome. When NPC1 is inactivated, sphingosine is the first lipid to be stored, suggesting that NPC1 plays a role in the transport of sphingosine from the lysosome, where it is normally generated as part of sphingolipid catabolism. Elevated sphingosine in turn causes a defect in calcium entry into acidic stores resulting in greatly reduced calcium release from this compartment. This then prevents late endosome-lysosome fusion, which is a calcium dependent process, and causes the secondary accumulation of lipids (cholesterol, sphingomyelin and glycosphingolipids) that are cargos in transit through the late endocytic pathway. Other secondary consequences of inhibiting NPC1 function include defective endocytosis and failure to clear autophagic vacuoles. It has been shown that the NPC1/NPC2 cellular pathway is targeted by pathogenic mycobacteria to promote their survival in late endosomes.

To facilitate administration, this invention also provides a composition containing one or more native, and/or synthetic peptides, and/or fragments thereof, and a pharmaceutically acceptable carrier or excipient. The pharmaceutical compositions provided herein can be formulated for oral, ocular, intravenous, intravitreal, subconjunctival, subcutaneous, intramuscular, intraperitoneal, intracerebral, intraarterial, intraportal, intralesional, intrathecal, or intranasal administration or topical administration. Suitable pharmaceutical compositions can be determined by one skilled in the art depending upon, for example, the intended route of administration, delivery format and desired dosage. See, for example, Remington's Pharmaceutical Sciences (19th edition, 1995).

The native and/or synthetic peptide(s) can be incorporated in a conventional dosage form, such as a gel, wash, cream, tablet, capsule, pill, solution, eye drop, spray, bandage, contact lens, depot, injectable, implantable, sustained-release formulation, or prolonged drug delivery system. The dosage forms may also include the necessary physiologically acceptable carrier material, excipient, lubricant, buffer, surfactant, antibacterial, bulking agent (such as mannitol), antioxidants (ascorbic acid or sodium bisulfite) or the like.

Acceptable formulation materials preferably are nontoxic to recipients at the dosages and concentrations employed. The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as PLURONICS, PEG, sorbitan esters, polysorbates such as polysorbate 20 and polysorbate 80, TRITON, trimethamine, lecithin, cholesterol, or tyloxapal); stability enhancing agents (such as sucrose or sorbitol); tonicity enhancing agents (such as alkali metal halides, preferably sodium or potassium chloride, mannitol, or sorbitol); delivery vehicles; diluents; excipients and/or pharmaceutical adjuvants. See, for example, Remington's Pharmaceutical Sciences, Id.

The primary carrier or excipient in a pharmaceutical composition may be either aqueous or nonaqueous in nature. For example, a suitable carrier or excipient may be water for injection, physiological saline solution or artificial cerebrospinal fluid, possibly supplemented with other materials common in compositions for parenteral administration. Neutral buffered saline or saline mixed with serum albumin are further exemplary excipients. Pharmaceutical compositions can include Tris buffer of about pH 7.0-8.5, or acetate buffer of about pH 4.0-5.5, which may further include sorbitol or a suitable substitute. Pharmaceutical compositions of the invention may be prepared for storage by mixing the selected composition having the desired degree of purity with optional formulation agents (Remington's Pharmaceutical Sciences, Id.) in the form of a lyophilized cake or an aqueous solution. Further, the peptides of the invention may be formulated as a lyophilizate using appropriate excipients such as sucrose.

Administration routes for the pharmaceutical compositions of the invention include the oral route; injection by intravenous, intraperitoneal, intracerebral (intra-parenchymal), intracerebroventricular, intramuscular, intra-ocular, intraarterial, intraportal, or intralesional routes; or via sustained release systems or by implantation devices. The pharmaceutical compositions may be administered by bolus injection or continuously by infusion, or by implantation device. The pharmaceutical composition also can be administered locally via implantation of a membrane, sponge or another appropriate material onto which the synthetic histatin(s) has been absorbed or encapsulated. Where an implantation device is used, the device may be implanted into any suitable tissue or organ, and delivery of the endogenous or synthetic histatin(s) may be via diffusion, timed-release bolus, or continuous administration.

When parenteral administration is contemplated, the compositions for use in this invention may be in the form of a pyrogen-free, parenterally acceptable aqueous solution containing the native and/or synthetic histatin(s) of the invention in a pharmaceutically acceptable vehicle. A particularly suitable vehicle for parenteral injection is sterile distilled water in which the peptide(s) is formulated as a sterile, isotonic solution, appropriately preserved. Preparation can involve the formulation of the peptide(s) with an agent, such as injectable microspheres, bio-erodible particles, polymeric compounds (such as polylactic acid or polyglycolic acid), beads or liposomes, that may provide controlled or sustained release of the peptide(s), which may then be delivered via a depot injection. In particular, formulation with hyaluronic acid has the effect of promoting sustained duration in the circulation.

The compositions may also be formulated for inhalation. In these embodiments, the peptide (s) of the invention is formulated as a dry powder for inhalation, or inhalation solutions may also be formulated with a propellant for aerosol delivery, such as by nebulization. Pulmonary administration is further described in, e.g., WO 1994/020069.

The pharmaceutical compositions of the invention can be delivered through the digestive tract, such as orally. The preparation of such pharmaceutically acceptable compositions is within the skill of the art. The peptide(s) of the invention that is administered in this fashion may be formulated with or without those carriers customarily used in the compounding of solid dosage forms such as tablets and capsules. A capsule may be designed to release the active portion of the formulation at the point in the gastrointestinal tract when bioavailability is maximized and pre-systemic degradation is minimized. Additional agents can be included to facilitate absorption of the synthetic peptide(s). Diluents, flavorings, low melting point waxes, vegetable oils, lubricants, suspending agents, tablet disintegrating agents, and binders may also be used.

These compositions may also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It may also be desirable to include isotonic agents such as sugars, sodium chloride and the like. Prolonged absorption of an injectable pharmaceutical form can be brought about by the inclusion of agents which delay absorption such as aluminum monostearate and gelatin.

In certain embodiments, a native and/or synthetic histatin peptide(s) is formulated in drop form; topical gel form; as a solid formulation (e.g., similar to LACRISERT, hydroxypropyl cellulose ophthalmic insert); by injection into the anterior chamber of the eye; by injection into posterior chamber of the eye for inhibition of angiogenesis, inhibition of destructive MMP activity or to enhance epithelial wound healing; by coating of surgical devices (intraocular lens, glaucoma device, keratoprosthetic, lacrimal intubation tubes, lacrimal bypass tubes); by coating of contact lenses; by coating of microbeads, nanobeads or other similar constructs; for systemic delivery; for delivery in mouth washes or gels; for delivery in topical applications through emulsions, creams, gels, ointments, or tinctures; long standing depot injections; triggered or delayed release formulations to oral, nasal, sinus, lung or upper airway mucosa; or rectal or transcatheteric (GI, GU, ostomy) formulations.

Suitable delivery methods further include conventional microparticle or nanoparticle delivery systems for penetrating the central nervous system or blood brain barrier, e.g., microparticles or nanoparticles composed of poly(lactide-co-glycolides), poly(lactides), or a lactic and glycolic acid (poly(lactic-co-glycolic acid)) copolymer (PLGA); PLGA nanoparticles with transferrin or lactoferrin surface modifications; nanoparticles densely coated with polyethylene glycol or a block copolymer containing polyethylene glycol blocks and having a near neutral charged surface; delivery systems based on cyclodextrins; and the like. See, e.g., WO 2020/210805 A1.

As one skilled in the art will also appreciate, the composition described herein can be formulated so as to carry a minimum of adverse side effects. The compositions described herein can be suitable for repeatable and long term use alone; useful as an adjunct therapy; and/or useful in a program involving rotation of agents, thereby decreasing long term exposure to (and, therefore, side effects resulting from) any one agent.

Given the newly identified role of TMEM97 and NPC1 pathways in wound healing and epithelial cell migration promoting activity in ocular tissue, this invention also provides methods of treating ocular diseases using agents that modulate the activity of TMEM97 and/or NPC1. Such agents include, but are not limited to, opipramol, MIN-101 (2-[[1-[2-(4-fluorophenyl)-2-oxoethyl]piperidin-4-yl]methyl]-3H-isoindol-1-one), CT-1812, siramesine, rimcazole, ibogaine, afobazole, BMY-14802 (1-(4-Fluorophenyl)-4-[4-(5-fluoro-2-pyrimidinyl)-1-piperazinyl]-1-butanol), and panamesine. The foregoing TMEM97 ligands are described, e.g., in German Federal Republic Patent No. 1,132,556, U.S. Pat. Nos. 9,458,130, 7,166,617, 8,765,816, PCT Publication No. WO 2015/116923, U.S. Pat. Nos. 5,665,725, 4,379,160, 4,499,096, Russian Patent No. 2,061,686, Russian Patent No. 2,485,954, U.S. Pat. Nos. 4,605,655, and 5,232,931. Additional exemplary TMEM97 ligands are ¹¹C-PB-28, ¹²⁵I-RHM-4, ¹²⁵I-IAC44, ¹²⁵I-IAF(1-N-(2′,6′-dimethyl-morpholino)-3-(4-azido-3-[(125)I]iodo-phenyl)propane, ¹⁸F-ISO-1, 2-(4-(3-(4-fluorophenyl)indol-1-yl)butyl)-6,7-dimethoxy-1,2,3,4-tetrahydroisoquinoline), ³H DTG, ³H-azido-DTG, ³H-PB28, ³H-RHM-1, ^{99m}Tc BAT-EN6, ^{99m}Tc-4-(4-cyclohexylpiperazine-1-yl)-butan-1-one-1-cyclopentadienyltricarbonyl technetium, ABN-1, AG-205, ANSTO-19, benzoxazolone, BIMU-1, CB-182, CB-184, CB-64D, CB-64L, cocaine, ditolylguanidine (DTG), F281, indole ((1-[3-[4-(substituted-phenyl) piperazin-1-yl]-propyl]-1H-indole, K05-138, K05-138, N-Benzyl-7-azabicyclo[2.2.1]heptane, PB183, PB28, RHM-1, RHM-138, RHM-2, RHM-4, SM-21, SN79, SV119, SW107, SW116, SW120, SW43, TC4ANSTO-19, WC-21, WC-26, WC-59, yun179, yun194, yun201, yun202, yun203, yun204, yun209, yun210, yun212, yun234, yun236, yun242, yun243 (RMH-1), yun245, yun250, yun251, yun253, yun254, and yun552. The foregoing ligands are described in, e.g., Guo & Zhen (2015) Curr. Med. Chem. 22(8):989-1003; and Mach, et al. (2013) J. Med. Chem. 56(18):7137-60. Further exemplary TMEM97 ligands include SAS-0132 (benzyl (1R,5S)-8-(4-methylpiperazin-1-yl)-1, 3, 4, 5-tetrahydro-2H-1, 5-methanobenzo [c] azepine-2-carboxylate), CM398 (1-(4-(6,7-Dimethoxy-3,4-dihydroisoquinolin-2 (1H)-yl) butyl)-3-methyl-1H-benzo[d]imidazol-2(3H)-one), LR-132, LR-172, MS-377, BIMU-1, RS-23597-190, BMS-181100, DKR-1051, DKR-1005, JVW-1009, and 2(3H)-benzoxalones and 2(3H)-benzothiazolones such as CM-156 and CM777 and any additional compounds described in Yi, et al. (2017) J. Neurochem. 140(4):561-575. Additional exemplary TMEM97 ligands include compounds 12, 16, 20, 39, 40, 19, 38, 27, 41, 42, 43, 44, 32, 33, 34, 35, 36, 37, 28, 29, 30, 31 (SAS-1121), and any additional compounds described in Sahn, et al. (2016) Chem Med Chem. 11(6):556-61. Additional TMEM97 ligands are disclosed in US Patent Application Publication No. 2006/0004036, US Patent Application Publication No. 2012/0190710, US Patent Application Publication No. 2013/0274290, PCT Publication No. WO 01/85153, PCT Publication No. WO 2001/80905, PCT Publication No. WO 1997/34892, PCT Publication No. WO 1997/30038, PCT Publication No. WO 1996/05185, EP Patent Publication No. 0881220, U.S. Pat. Nos. 6,015,543, 5,993,777, 5,919,934, 5,969,138, 5,911,970, and PCT Publication No. WO 2001/85153. Additional ligands are known and described in Huang, et al. (2014) Med. Res. Rev. 34(3):532-66; Cheng et al. (2020) Curr. Med. Chem. 27:1-18; Floresta, et al. (2018) Marine drugs 16(10):384; Rescifina, et al. (2017) Data Brief 13:514-35; Nastasi, et al. (2017) J. Cheminformatics 9(1):1-9); Alon, et al. (2021) bioRxiv 2021.04.29.441652; and the S2RSLDB database. In some embodiments, agents the modulate TMEM97 activity, antagonize TMEM97. In other embodiments, agents that modulate TMEM97 activity, agonize TMEM97.

Ocular diseases that can be treated using an effective amount of one or more the above-referenced TMEM97 ligands include, but not limited to, ocular inflammation, ocular wound healing, corneal wound healing, conjunctival wound healing, retinal degeneration, diabetic retinopathy, age related macular degeneration, corneal neuropathies (including diabetic neuropathy), dry eye disease (evaporative, aqueous deficient or others) Sjogren's syndrome, ocular graft versus host disease, glaucoma (primary, secondary, congenital, adolescent, traumatic, inflammatory), uveitis, bacterial infections, viral infections, fungal infections, scleritis, orbital inflammatory or infectious syndromes, thyroid eye disease, strabismus, conjunctivitis, ocular surface disorders, allergic and atopic eye diseases, Meibomian gland disorders and rosacea, amongst others.

Synthetic peptides including the SHRGY (SEQ ID NO:31) sequence have also been shown to increase ERK1/2 activation. Accordingly, the present invention also provides a method for increasing ERK activation by administering to a subject in need of such treatment one or more TMEM97 modulators in an amount effective to increase ERK activation. It is well established that ERK modulation is important in both the innate and adaptive immune systems (Zhang & Dong (2005) Cell. Mol. Immunol. 2(1):20-27.

In this context of the methods therein, a “subject” is meant to include humans, as well as non-human animals. As used herein, the term “effective amount” or “therapeutically effective amount” refers to an amount of agent disclosed herein (e.g., histatin peptide or TMEM97 modulator) or a pharmaceutical composition containing the same sufficient to achieve the stated desired result. In some aspects, an effective amount provides a measurable improvement in, e.g., a lysosomal storage disease such as Neimann Pick Type C Disease or phenotype, or ocular disease or phenotypes (e.g., the rate epithelial cell migration, the rate or time to wound closure, and/or an increase in ERK and survival pathway modulation), as compared to a subject that has not received such treatment. The amount of the agent which constitutes an “effective amount” or “therapeutically effective amount” may vary depending on the severity of the disease, the condition, weight, or age of the patient to be treated, the frequency of dosing, or the route of administration, but can be determined routinely by one of ordinary skill in the art. Depending on the location and condition to be treated, a dose in the range of 1 picomolar to 500 molar or more of the agent may be used. A clinician may titer the dosage or route of administration to obtain the optimal therapeutic effect. Typical dosages range from about 0.1 μg/kg to up to about 100 mg/kg or more, depending on the factors mentioned above. In certain embodiments, the dosage may range from 0.1 μg/kg up to about 100 mg/kg, or 1 μg/kg up to about 100 mg/kg, or 5 μg/kg up to about 100 mg/kg.

“Treating” a subject means accomplishing one or more of the following: (a) reducing the severity of the disease or condition; (b) arresting the development of the disease or condition; (c) inhibiting worsening of the disease or condition; (d) limiting or preventing recurrence of the d disease or condition in patients that have previously had the disease or condition; (e) causing regression of the disease or condition; (f) improving or eliminating the symptoms of the disease or condition; and/or (g) improving survival.

The following non-limiting examples are provided to further illustrate the present invention.

### Example 1: Materials and Methods

Peptide Synthesis and Purification. Histatin 1 (Hst1), Hst1 scrambled peptide (Hst1SP) and TMEM97 peptides [TMEM97 (108-176), TMEM97 (108-143), TMEM97 (144-176)](Table 6) were synthesized according to known methods.

TABLE 6

| | | SEQ ID |
|---|---|---|
| Name | Sequence | NO: |
| Hst1 | DpSHEKRHHGYRRKFHEKHHSHREFPFYGDYGSNYLYDN | 6 |
| Hst1SP | HYHKFHRYYDPGSNLYKEHNHGFHHGYKDEFRREpSRDS | 71 |
| TMEM97 | MTTLIPILSTFLFEDFSKASGFKGQRPETLHERLTLVSV | 72 |
| (108-176) | YAPYLLIPFILLIFMLRSPYYKYEEKRKKK | |
| TMEM97 | MTTLIPILSTFLFEDFSKASGFKGQRPETLHERLTL | 73 |
| (108-143) | | |
| TMEM97 | VSVYAPYLLIPFILLIFMLRSPYYKYEEKRKKK | 74 |
| (144-176) | | |

″Sp″ or ″S(PO₃)″ denotes phosphorylated serine.

Briefly, linear peptides were synthesized using the stepwise solid-phase method by the 9-fluorenylmethoxycarbonyl (Fmoc) chemistry on the Wang resin (AnaSpec; Fremont, Calif.) with a channel multiplex peptide synthesizer (Protein Technologies; Tucson, Ariz.) according to the manufacturer's procedures. Peptide synthesis started from the C-terminus of the peptide. The Fmoc group of the resin was removed with 20% piperidine in N,N-dimethylformamide (DMF) (5 minutes, X2) followed by washing the resin with DMF (30 seconds, 6×) before the amino acid (Fmoc protected, 2 equiv) was added in the presence of 0.2 M 2-(1H-Benzotriazole-1-yl)-1,1,3,3,-tetramethyluronium hexafluorophosphate (HBTU, 1.9 equiv) and 0.4 M 4-methylmorpholine (NMM, 4 equiv) in DMF (30 minutes, X3). Excess reagents were washed away (30 seconds, 6×) with DMF. The process was repeated until the last amino acid was added. After completion, the N-terminal Fmoc was removed with 20% piperidine in DMF (5 minutes, X2) followed by washing the resin with DMF (30 seconds, 6×). Detachment of peptide from the resin and removal of the side chain protection groups were done by incubating the resin with triflouroacetic acid (TFA):Thioanisole:Water:Phenol:1,2-ethanedithio (82.5:5:5:5:2.5 v/v) cocktail for 2 hours. The reaction mixture was filtered followed by washing the resin with TFA (2×). Ice-cold ethyl ether was added to precipitate the peptide and the pellet was washed 2 times with ice-cold ethyl ether. The crude peptide was subsequently dissolved in 50% acetonitrile in water and lyophilized.

The crude peptide was purified on a preparative KINETEX® reversed-phase C18 column, 150×21.1 mm (Phenomenex; Torrance, Calif.) using a BioCad Sprint™ HPLC system (Applied Biosystems; Foster City, Calif.). A flow rate of 30 mL/minute with solvent A (0.1% TFA in deionized water) and solvent B (0.1% TFA in acetonitrile) was used. The column was equilibrated with 5% solvent B before sample injection. Elution was performed with a linear gradient from 5% solvent B to 100% solvent B in 60 minutes. The absorbance of the column effluent was monitored at 214 nm, and peak fractions were pooled and lyophilized. The pure peptide fraction was identified by electrospray ionization mass spectrometry (ESI MS) and lyophilized.

Radioligand Binding Assay. Radioligand binding/competition assays were performed by the University of North Carolina (UNC) Psychoactive Drug Screening Program (PDSP). An S2R transient overexpression HEK293T cell line was used for membrane preparations. Primary and secondary radioligand binding assays were then performed using an initial 10 μM concentration of Hst1 followed by determination of equilibrium binding affinity over multiple concentrations, in triplicate. The “hot ligand” for S2R was [3H]-1,3-di-o-tolylguanidine ([3H]-DTG) and haloperidol was used as the prototypical inhibitor. Calculations of the percentage inhibition for each assay plate with total binding (with buffer) as 0% inhibition and nonspecific binding (in the presence of the reference compound) as 100% inhibition over an average of four experiments are used to identify compounds suitable for secondary screening (>50% inhibition). Secondary screening results are reported as amount of hot ligand binding [counts per minute (CPM)] remaining with a standard reference dose-response curve (all in triplicate). Determination of Ki's for the reference drug (haloperidol) and the experimental article (Hst1) is then performed. Secondary screening assays are performed three separate times with three technical replicates for each experiment.

Cell Culture. Human corneal epithelial (HCE) cells were provided by Deepak Shukla (University of Illinois at Chicago; Chicago, Ill.). HCE cells were cultured in a Medium Essential Media (MEM) (Corning, Cellgro; Manassas, Va.) supplemented with 10% Fetal Bovine Serum [(FBS), Gibco Life Technologies; Grand Island, N.Y.)] and 1% penicillin. Standard cell culture conditions (37° C., 5% CO₂, >95% humidity) were used during routine passages.

Immunoprecipitation/Western Blot Analysis. The day before, HCE cells were plated at the concentration of 5×10⁶ cells/well in a 100 mm dish and were treated with 20 μM of Hst1 for 6 hours. Cells were harvested with lysis buffer (1% NP40, 137 mM sodium chloride, 20 mM Tris [pH 8.0], and 10% glycerol) and the lysates were incubated with 5 μL of a validated rabbit polyclonal anti-TMEM97 antibody (Novus Bio.; Littleton, Colo.) overnight at 4° C. The lysates were incubated with 30 μL of a suspension of protein A/G (Santa Cruz Biotechnology; Dallas, Tex.) for 2 hours at 4° C. with gentle shaking. After centrifugation for 5 minutes, pellets were washed three times and resuspended in 50 μL of 2×NUPAGE® LDS sample buffer (Invitrogen; Carlsbad, Calif.) and boiled for 10 minutes.

For detecting Hst1 bound to TMEM97 protein, the lysates were subjected to electrophoresis on 12% NUPAGE® Bis-Tris gels (Invitrogen; Carlsbad, Calif.), followed by transfer to nitrocellulose membranes (Amersham Protran, GE Healthcare; Pittsburgh, Pa.). Membranes were then blocked with Tris-buffered saline containing 3% nonfat dry milk for 1 hour and incubated with rabbit primary antibody against Hst1 (Mybiosource; San Diego, Calif.) (1:1000) overnight at 4° C. After washing in 0.05% Tris-buffered saline containing 0.05% polysorbate 20 sold under the tradename TWEEN® 20 (TBST), membranes were then incubated for 1 hour with goat anti-rabbit-HRP (BD Biosciences; San Jose, Calif.) (1:2000) as the secondary antibody. The membranes were developed using MYELC Imager (Thermo Fisher Sci.; Waltham, Mass.) and ECL Pro solution (PerkinElmer; Waltham, Mass.). β-actin was used as an internal control.

Circular Dichroism (CD). CD analysis was performed on a Jasco 815 CD spectrometer at room temperature. TMEM97 (108-176) and Hst1 full-length peptides were prepared as 10 mM stock in 25% DMSO and water, respectively, and diluted to 0.15 mg/mL final concentration in 10 mM Na₃HPO₄ buffer. A total of 400 μL of each sample was added into a 1 mm quartz sample cell, and CD spectra were recorded from 260 nm to 190 nm wavelength. Data points were measure in 0.5 nm wavelength step at a scanning speed of 100 nm/minute. A total of five spectra were acquired for each sample and averaged. Na₃HPO₄ buffer without peptide was used as a control curve, which was subsequently subtracted from the CD spectra of peptide samples. The resulting buffer control subtracted CD intensity row data in millidegrees were submitted to DichroWeb and fitted with multiple embedded models and converted to mean residue ellipticity.

Surface Plasmon Resonance (SPR). Recombinant full-length TMEM97 (also called MAC30, from HEK293 cells) and GST-TMEM97 (108-176, recombinant GST-C terminal from wheat germ) proteins were purchased from OriGene (Rockville, Md.) and Abnova (Taipei City, Taiwan), respectively. Three peptides, TMEM97 (108-176), TMEM97 (108-143) and TMEM97 (144-176), were synthesized. All proteins and peptides were initially prepared in HBS buffer containing 10 mM HEPES, pH 7.4, 150 mM NaCl, and 0.05% surfactant P20. The CM5 sensor surface was first activated by 1-ethyl-3-(3-dimethylaminopropyl) carbodiimide hydrochloride (EDC)/N-hydroxy succinimide (NHS) mixture using a Biacore T200 or Biacore 8K instrument (GE Healthcare). Two recombinant proteins, full-length TMEM97 and GST-TMEM97 (108-176), were diluted to 50 μg/mL in 10 mM sodium acetate at pH 5.5 and immobilized to flow channels 2 and 4 followed by ethanolamine blocking on the unoccupied surface area. Flow channels 1 and 3 were used as references.

The three synthesized peptides were diluted to 50 μg/mL in 10 mM sodium acetate at pH 4.0 and immobilized to flow channels 2, 3 and 4, respectively, on another sensor chip. The unmodified surfaces on a flow channel 1 were used as a reference control. Histatin solutions with a series of increasing concentrations (0.78-25 μM at 2-fold dilution) were applied to all four channels at a 30 μL/minute flow rate at 25° C. The data were double-referenced with reference channel and zero concentration responses, and reference subtracted sensorgrams were fitted with 1 to 1 Langmuir kinetic model using a Biacore T200 evaluation software V3.0 or Insight evaluation software. The equilibrium dissociation constants (K&) were determined from two rate constants (K_{D}=k_{d}/kₐ).

Isothermal Titration Calorimetry (ITC). The three synthesized peptides, TMEM97 (108-176), TMEM97 (108-143) and TMEM97 (144-176) were prepared in ITC buffer (10 mM Tris, pH 7.4, 150 mM NaCl, 0.05% polysorbate 20) at 40 μM concentration and placed in the sample cell. All ITC experiments were performed while stirring at 395 rpm, in ITC buffer at 25° C. using a VP-ITC titration microcalorimeter from MicroCal™, LLC (Northampton, Mass.). The microsyringe was loaded with a Histatin 1 solution (500 μM in ITC buffer). All titrations were conducted using an initial injection of 2.5 μL followed by 35 identical injections of 5 μL with a duration of 16 seconds (per injection) and a spacing of 210 seconds between injections. The buffer control titration (Histatin 1 into buffer) signals were subtracted from the experimental data. The collected data were evaluated using NITPIC (NIH), SEDPHAT (NIH), and GUSSI (NIH).

Immunofluorescence Imaging. HCE cells were seeded on glass coverslips (Fisher Scientific Co.; Pittsburgh, Pa.) within a 6-well plate at 3×10⁵ (cells/well) seeding density. The cells were washed with media and were treated 20 μM of Hst1 or untreated, both with reduced serum conditions (0.5% FBS in MEM media) for 6 hours. HCE cells were then fixed in 3.7% paraformaldehyde, permeabilized with phosphate-buffered saline (PBS) containing 0.2% TRITON™ X-100 for 5 minutes, and washed three times for 5 minutes each time in PBS. For blocking, cells were and incubated at room temperature for 30 minutes with 5% bovine serum albumin (BSA) and 5% normal goat serum in PBS. For the Endoplasmic Reticulum (ER) staining, a Cytopainter ER staining kit (Abcam; Cambridge, Mass.) was used, following the manufacturer's instructions. After washing with 1×Assay buffer, cells were incubated with Green Detection Reagent to cover the monolayer of cells. For the detection of Hst1 and TMEM97, mouse anti-Hst1 antibody (Abcam; Cambridge, Mass.), rabbit primary antibody against Hst1 (Mybiosource; San Diego, Calif.), and rabbit anti-TMEM97 antibody (Novus Bio.; Littleton, Colo.) were used. Cells were incubated overnight at 4° C. with primary antibodies, washed three times for 5 minutes each time in PBS before incubation with secondary antibodies for 30 minutes. Cells were then counterstained with 1 μg/mL 4′,6-diamidino-2-phenylindole (DAPI) solution in PBS for 3 minutes, then washed three times with PBS containing 0.05% TRITON™ X-100 for 5 minutes each time, twice with PBS for 5 minutes each time, and once with distilled water for 10 seconds. The cells were mounted in Fluoro gel with Tris buffer (Electron Microscopy Sciences; Hatfield, Pa.) and observed under a confocal microscope (Zeiss LSM 710 Confocal Microscope; Oberkochen, Germany) using a 40×objective. Fluorescent dye sold under the tradename ALEXA FLUOR® 488 NHS Ester (Molecular Probes; Carlsbad, Calif.) was used for making Alexa-488 coupled synthetic Hst1.

Transfection/Knock-Down of TMEM97. Sub-confluent monolayers of HCE cells in grown in 35-mm six-well plates were transfected with reaction mixtures composed of 100 pmol of small interfering RNA (siRNA) to TMEM97 (Santa Cruz Biotechnology; Dallas, Tex.) for 48 hours and 5 μL of transfection reagent sold under the tradename LIPOFECTAMINE® 2000 (Invitrogen; Carlsbad, Calif.) in culture medium sold under the tradename OPTI-MEMO media (Gibco Life Technologies; Grand Island, N.Y.). Complexes were incubated for 20 minutes at 24° C. and then added to cells at 37° C. Incubation was continued for 24 to 48 hours at 37° C. in 5% CO₂. Knock-down (KD) of TMEM97 was confirmed by western blot analysis using a rabbit anti-TMEM97 antibody (Novus Bio.; Littleton, Colo.).

Cell Migration Assay. HCE migration assays were performed in a 48-well micro-chemotaxis chamber (Neuro Probe, Inc.; Cabin John, Md.), following the manufacturer's instructions and following a modification of prior reports. Polyester membranes (Neuro Probe, Inc.; Gaithersburg, Md.) with 12 μm pores were used. Cells were incubated with Hst1 for 6 hours, harvested using Versene (Life Technologies, Corp.; Grand Island, N.Y.), resuspended in RPMI-1640 medium (Life Technologies, Corp.; Grand Island, N.Y.) containing 0.5% FBS. The bottom chamber was loaded with RPMI-1640 media containing 2% FBS, and the filter was laid over the media. The upper chamber was loaded with 3×10⁴ cells and then incubated at 37° C. for 16 hours. The filters were then fixed and stained using Eosin (Richard-Allan Sci.; Kalamazoo, Mich.). Each condition was studied in triplicate wells, and each experiment was performed three separate times, with three replicates from a single experiment.

Wound Healing in vitro Scratch Assay. HCE cells were cultured in a 96-well plate at 5×10⁴ (cells/well) seeding density, and were grown to confluence. Subsequently, a straight line scratch mark was made with a multiscratch wound maker (IncuCyte® 96-well WoundMaker Tool, Essen Biosciences; Ann Arbor, Mich.). The cells were then washed twice with PBS to remove cellular debris. Wounded areas were then treated with or without 20 μM or 50 μM of Hst1 in reduced serum conditions (0.5% FBS). Scratches were photographed microscopically at 4× magnification (Image express Micro, Molecular devices; San Jose, Calif.) every hour over the course of the experiment. The wound areas were measured using ImageJ software (ImageJ 1.47v, NIH, Thornwood; Bethesda, Md.). Relative wound closure was calculated by dividing the closure of the treated wound by that of the untreated control wound. Each condition was studied in triplicate wells, and each experiment was performed three separate times, with three replicates from a single experiment.

### Example 2: Binding Assays Demonstrate that Hst1 Binds to TMEM97

Screening for potential receptors for Hst1 was carried out using radioligand binding assays on an existing library of overexpressed cell lines of various pharmacologically important receptors. Binding of synthetic Hst1 with TMEM97 containing HEK293T membranes was identified in a primary binding screen and confirmed with a secondary binding assay. Results were compared with a gold-standard inhibitor, haloperidol, over a range of doses as described. Results of the secondary radioligand binding assay confirmatory test demonstrated a Kᵢ for Hst1 of 239 nM as opposed to gold standard haloperidol (Kᵢ=44 nM), indicating the pharmacologic relevance of this interaction. In similar experiments, binding of Hst3 and Hst5 with TMEM97 containing HEK293T membranes was determined in the radioligand binding assay. The results of this analysis indicated that Hst3 and Hst5 also bound to TMEM97 with Kᵢ values of 1088 nM and 582 nM, respectively.

A co-immunoprecipitation (co-IP) assay was performed to determine if the interaction between Hst1 and TMEM97 was reproducible at the cellular level. After exogenous application of Hst1 to HCE cells, cell lysates were obtained and immunoprecipitation with a TMEM97 antibody followed by immunoblotting with an Hst1 antibody demonstrated that Hst1 was co-precipitated with TMEM97. Interestingly, two bands (one just below 10 kDa and one just below 15 kDa) were noted on co-IP. These bands may represent monomeric and dimeric versions of Hst1 and are similar to what has been seen with western blot analyses of Hst1 containing samples. These results indicate that the noncellular findings of this interaction are demonstrable in the physiologic cellular environment. Enzyme-linked immunosorbent assay (ELISA) were also conducted to detect bound Hst1 on plates coated with TMEM97. This analysis further confirmed binding between Hst1 and TMEM97. Notably, addition of the prototypical TMEM97 agonist, 1,3-Di-o-tolylguanidine (DTG), did not alter Hst1 binding to TMEM97 in ELISA, indicating a novel interaction site.

Subsequently, the hypothesis that interaction between Hst1 and TMEM97 could affect the structure of each protein was tested. Also, the secondary structure of Hst1 and TMEM97 was tested in solution using CD measurements. It was noted that Hst1 alone is mostly disordered (over 50%) with some degree of β-strand, while TMEM97 (108-176) contains some α-helical and β-strand regions with ˜34% disordered regions. According to CD spectra comparison, there seemed to be more secondary structures formed upon Hst1 binding to TMEM97 (108-176). CD data analysis using DichorWeb revealed that disordered regions of the Hst1-TMEM97 (108-176) complex were significantly lower than each of the proteins alone (Table 7). These results indicate that not only does Hst1 bind to TMEM97, but the interaction may further induce secondary structures.

TABLE 7

| Protein | Helix | Strand | Turns | Unordered |
|---|---|---|---|---|
| TMEM97 (108-176) | 0.14 | 0.31 | 0.21 | 0.34 |
| Hst1 | 0.02 | 0.29 | 0.15 | 0.54 |
| Hst1 + TMEM97 (108-176) | 0.18 | 0.42 | 0.26 | 0.14 |

SPR testing was performed and demonstrated that Hst1 selectively bound to full-length recombinant TMEM97 produced in a eukaryotic system (HEK293 cells; K_{D}=1.3±0.3 μM, % Rₘₐₓ=32%). Subsequent testing with a GST-tagged, wheat-germ derived, C-terminal fragment TMEM97 (108-176) determined that Hst1 binding to the C-terminal region of TMEM97 was possible with similar affinity (K_{D}=1.6±0.2 μM, % Rₘₐₓ=43%). A scrambled peptide control version of Hst1 did not exhibit significant binding to either full-length TMEM97 (No binding, % Rₘₐₓ=8%) or TMEM97 (108-176) (No binding, % Rₘₐₓ=5%). These results indicate that there is specific binding of Hst1 with the C-terminus of TMEM97, and that this interaction is reproducible with multiple different recombinant sources of TMEM97.

To determine more precisely which segment of TMEM97 is necessary for binding of Hst1, multiple synthetic fragments of TMEM97 were generated (Table 6) and binding to Hst1 was tested using SPR. It was found that the full C-terminal synthetic TMEM97 (108-176; K_{D}=2.8±0.6 μM, % Rₘₐₓ=38%) bound Hst1 with similar affinity to full-length and GST-tagged recombinant TMEM97. Smaller fragments of the C-terminal of TMEM97 were then constructed to represent the (predicted) luminal (108-143) and cytoplasmic (144-176) exposed segments of the C-terminus of TMEM97 based on modeling experiments previously described. The results of this analysis indicated that the TMEM97 (144-176) was necessary for Hst1 binding (K_{D}=2.7±0.4 μM, % Rₘₐₓ=21%), and that TMEM97 (108-143) was unable to bind Hst1. In similar SPR analysis, Hst3 and Hst5 were found to bind full length TMEM97 and GST-TMEM97 (108-176) with K_{D} values of 3.8 μM (% Rₘₐₓ=56%) and 4.7 μM (% Rₘₐₓ=72%), respectively. Notably, mutations of the presumed binding sites of DTG for TMEM97 (i.e., mutations E170A, E171A and E170A/E171A) did not affect Hst1 binding to TMEM97 (144-176), indicating a novel interaction site. Moreover, addition of either DTG or haloperidol to the reaction did not affect binding of Hst1 to either full length TMEM97 or GST-TMEM97 (108-176). SPR results for Hst1 were confirmed using ITC as an orthogonal analytical method. Similarly, it was found that Hst1 bound TMEM97 (108-176) with high affinity (K_{D}=0.89±0.2 μM, ΔH=−4.9±10.1 kcal/mol, ΔS=11.6±3.4 cal/mol K) and that the C-terminal of TMEM97 was necessary for this interaction (K_{D}=0.46±0.12 μM, ΔH=−790±28 kcal/mol, ΔS=−2.28(±0.37)×10³ cal/mol K). Taken together, these results indicate that Hst1 specifically binds to TMEM97, and that the residues 144-176 of TMEM97 are necessary for this interaction.

To determine whether binding assay results were relevant to normal cellular conditions and function, several assays were performed to confirm the existence and relevance of an interaction between Hst1 and TMEM97. Testing was conducted to determine whether Hst1 could be internalized into HCE cells and whether localization of internalized Hst1 could colocalize with TMEM97. Using a fluorescent dye-coupled synthetic Hst1, it was demonstrated that exogenous application of this peptide was internalized into HCE cells at 24 hours after exposure, with relative enrichment of localization to the peri-nuclear area. Subsequent immunolocalization of exogenously applied Hst1 to HCE cells and visualization with an endoplasmic reticulum staining agent demonstrated good co-localization. Finally, co-immunolocalization of exogenously applied Hst1 with TMEM97 demonstrated significant overlap. These results indicate that Hst1 is localized to the area where TMEM97 is thought to have a functional role, and that the previously described binding assays may have a physiological correlate in live cells.

### Example 3: TMEM97 Mediates Hst1 Induced HCE Migration and Wound Healing

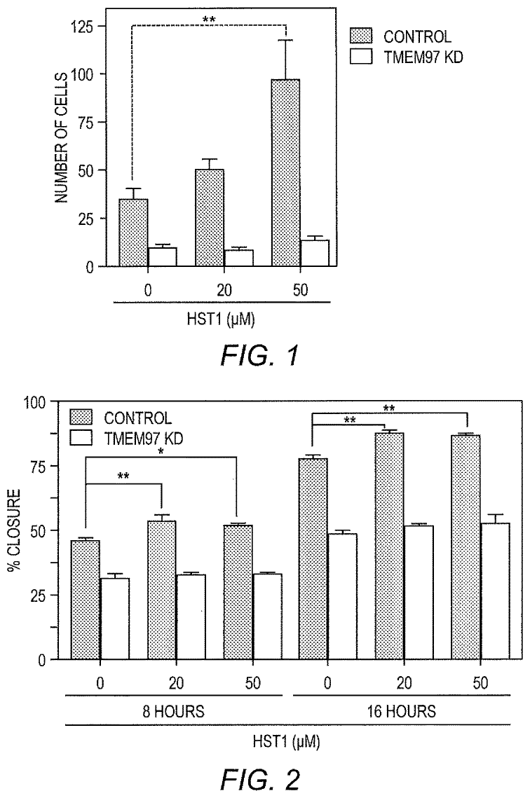

Testing was then performed to determine if the known functions of Hst1 on HCE were dependent upon TMEM97, including cell migration and wound healing. In order to assess the importance of TMEM97 in the known functions of Hst1, an siRNA knock-down (KD) of TMEM97 was performed in HCE cells. Internalization and localization of Hst1 in KD cells versus wild-type cells was then tested. Notably, KD of TMEM97 significantly disrupted internalization and/or localization of Hst1 in KD cells. Boyden chamber-based cell migration assays were performed and demonstrated that Hst1 treatment caused a dose-dependent increase in cellular transmigration toward a stimulus (2% FBS). KD of TMEM97 in HCE cells abolished this response to Hst1, indicating that transmigration acceleration in response to Hst1 is dependent upon the presence of TMEM97 (FIG. 1). Subsequently, it was tested whether Hst1 responsive increases in wound healing of HCE cells were dependent upon TMEM97. Using a standard scratch assay, it was found that increases in wound healing rates were noted in response to Hst1 in HCE cells. siRNA KD of TMEM97 abolished this responsiveness (FIG. 2). These findings indicate a novel pathway/mechanism for Hst1 induced wound healing and epithelial cell migration.

### Example 4: N-Terminus of Hst1 is Required for TMEM97 Binding

To determine the region(s) of Hst1 that mediate binding to TMEM97, SPR testing was performed with various Hst1 fragments and either recombinant TMEM97 (GST-tagged, wheat-germ derived, C-terminal fragment TMEM97 (108-176)) or synthesized TMEM97 (TMEM97 (108-176), TMEM97 (108-143) and TMEM97 (144-176)). The results of these analyses are presented in Table 8.

TABLE 8

| | | Synthetic | Synthetic | Synthetic |
|---|---|---|---|---|
| | GST-TMEM97 | TMEM97 | TMEM97 | TMEM97 |
| Hst* | (108-176) | (108-176) | (108-143) | (144-176) |
| Hst1(1-38) | K_{D} = 1.5 μM | K_{D} = 2.4 μM | No binding | K_{D} = 2.4 μM |
| | (% Rmax = 23%) | (% Rmax = 23%) | | (% Rmax = 22%) |
| Hst1(1-26) | K_{D} = 2.1 μM | K_{D} = 1.3 μM | No binding | K_{D} = 1.7 μM |
| | (% Rmax = 24%) | (% Rmax = 8%) | | (% Rmax = 4%) |
| Hst1(1-19) | K_{D} = 3.6 μM | K_{D} = 12.7 μM | Not tested | K_{D} = 8.8 μM |
| | (% Rmax = 32%) | (% Rmax = 19%) | | (% Rmax = 16%) |
| Hst1(1-14) | Not tested | No binding | No binding | No binding |
| Hst1(20-32) | Not tested | Not tested | Not tested | No binding |
| Hst1(15-38) | No binding | No binding | Not tested | No binding |
| Hst1(1-9 | No binding | No binding | Not tested | No binding |
| SP) | | | | |

*Residues included in Hst1 fragment are in parenthesis.
SP, scrambled peptide.

These results indicate that binding of Hst1 with the C-terminus of TMEM97 is mediated by the N-terminus of histatin, residues DSHAKRHHGYKRKFHEKHH (SEQ ID NO:75).

### Example 5: Histatins Modulate Cellular Metabolism

Cholesterol. To demonstrate to use of histatins in modulating the activity of NPC1, NPC1 cells were treated with Hst1 (20 μM or 50 μM) with or without siRNA-mediated knockdown (KD) of NPC1. Filipin staining, the generally accepted tool for detection of cholesterol deposits in NPC cells (Vanier, et al. (2003) Clin. Genet. 64:269-81), was used to visualize cholesterol in control and NPC1 KD cells. This analysis indicated increased clustering and collection of filipin-stained cholesterol in NPC1 KD cells, consistent with the NPC1 phenotype. By comparison, treatment with Hst1 reduced cholesterol accumulation and reduced the findings of NPC1 disease.

It was further demonstrated that Hst1 peptide treatment of NPC1 patient fibroblasts alters cholesterol metabolism by increasing NPC1 gene expression and reducing HMG-CoA reductase gene expression. In addition, cholesterol accumulation in wild type human skin fibroblasts or NPC knockout fibroblasts (NPC1^{-/-}) were evaluated with or without Hst1 treatment. This analysis indicated a reduction in cholesterol accumulation in Filipin stained cells and normalization of the NPC disease phenotype to near normal levels. Additionally, LAMP1 lysosomal marker accumulation in untreated NPC1^{-/-} cells were normalized with Hst1 treatment. Taken together, these results indicate that histatin peptides are of use in the treatment of NPC disease in addition to other lysosomal storage disorders.

Hst1 peptide treatment of mouse cerebellar tissue (7-week-old NPC mouse model) was also found to alter cholesterol metabolism by significantly increasing gangliosides (GM3, GM2 and GM1; p<0.001) and ceramides (Cer NS (d18:1/16:0; p-0.05) HexCer NS (d36:1; p=0.05) and SM (d34:0; p<0.001)). Moreover, in NPC1^{-/-} mouse cerebellar tissue (from a 9-week-old mouse), Hst1 treatment was found to significantly decrease phosphatidylinositol 4,5-bisphosphate (PIP₂; 20:4/18:0) levels (p<0.001). In this respect, in addition to reducing the accumulation of cholesterol in liver tissue, histatin peptides may also be used to alter fatty acid and cholesterol metabolism in neuronal tissue thereby reducing changes associated with neurodegeneration.

Autophagy. NPC1-deficiency results in increased autophagy as evidenced by elevated microtubule-associated protein 1 light chain 3 (LC3) levels, numerous autophagic vacuoles and enhanced degradation of long-lived proteins (Pacheco, et al. (2007) Human Mol. Genet. 16(12):1495-1503). Consistent with previous findings, immunolocalization of LC3 in NPC1 KD cells showed increases in LC3 staining indicative of NPC1 disease. Treatment of these cells with Hst1 (20 μM or 50 μM) provided a dose-dependent reduction in LC3 levels indicating that Hst1 reduces the effects of NPC1 deficiency and is therefore of use in the treatment of NPC1 disease.

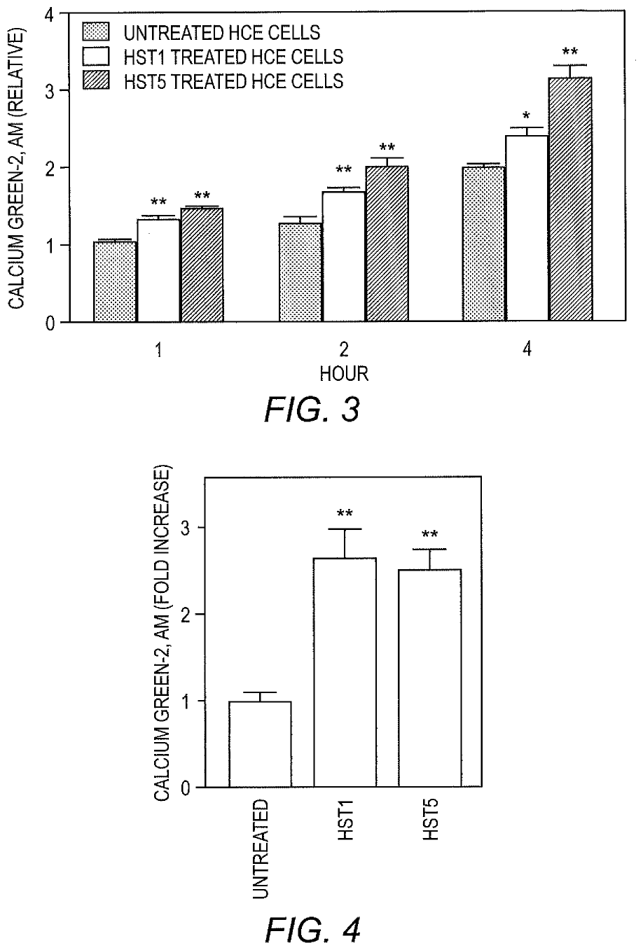

Calcium Signaling. Human corneal epithelial cells were treated with Hst1 or Hst5, stained with Calcium Green-2, and relative fluorescence intensity was monitored at 1, 2 and 4 hours. This analysis indicated that histatin peptide treatment increases calcium signaling in human corneal epithelial cells at each time point (FIG. 3), which is consistent with an agent targeting the Sigma-2 receptor. Similar increases in calcium signaling were observed in human skin fibroblasts treated with Hst1 and Hst5. Moreover, Hst1 treatment of NPC1 (I1061T mutant) homozygous patient fibroblasts increased cellular calcium levels (FIG. 4). However, Hst1 and Hst5 peptides reduced elevated calcium levels in human corneal epithelial cells treated with benzalkonium chloride (0.001% BAK). This normalization of cellular calcium levels seen after toxicity from benzalkonium chloride treatment is analogous to toxic effects seen in chronic pain and other conditions. As such, histatin peptides may of use in the amelioration of chronic pain.

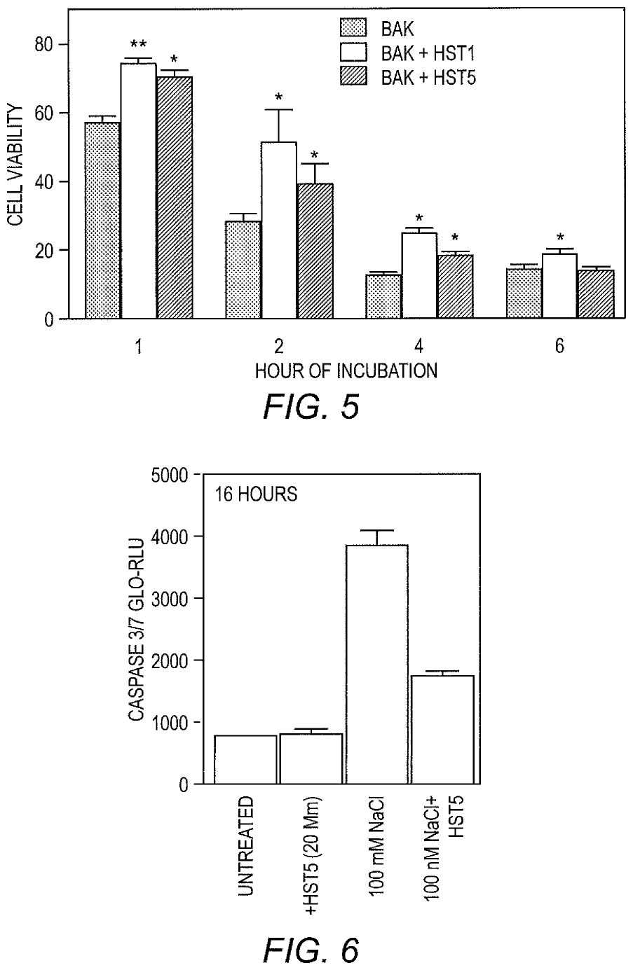

Apoptosis. Human corneal epithelial cells treated with BAK exhibit an induction of cell death and/or loss of cell viability. Notably, this loss cell viability is abrogated by treatment of these cells with Hst1 or Hst5 (FIG. 5). Similarly, human corneal epithelium exhibit cell death induced by toxic hyperosmolarity (hOsm). However, treatment of these cells with Hst5 abrogates the effects of toxic hyperosmolarity (i.e., 450 mOsm) and induction of apoptotic signals such as Caspase 3/7 (FIG. 6). BAK and hOsm are standard inducers of corneal epithelial and neuronal cell death and toxicity, phenomena which are associated with ocular surface pain and chronic pain syndromes. Taken together, these results indicate a therapeutic application for histatin peptides in chronic pain syndromes.

The calcium hypothesis of neurodegeneration suggests that abnormal calcium dynamics are associated with neurodegeneration. It is also thought that NPC disease is associated with reductions in cellular calcium and that mobilization of calcium could be associated with cholesterol normalization and reduction in neurodegeneration. Accordingly, the findings presented herein provide support for the use of histatin peptides in the treatment of multiple neurodegenerative disorders including Alzheimer's disease and traumatic brain injury, as well as in treatment of chronic pain. Notably, when tested in a human liver microsomal assay, the metabolic rate of Hst1 was similar to that of diphenhydramine (positive control), thereby demonstrate that histatin peptides can be dosed in an acceptable manner for treatment of human disease systemically without rapid degradation.

What is claimed is:

1. A method of treating a lysosomal storage disorder comprising administering to a subject in need of treatment an effective amount of one or a combination of histatin peptides to treat the subject's lysosomal storage disorder.

2. The method of claim 1, wherein the effective amount of the one or combination of histatin peptides reduces the accumulation of cholesterol, modulates calcium signaling, decreases apoptotic signaling, reduces losses in cell viability, or reduces microtubule-associated protein 1 light chain 3 protein.

3. The method of claim 1, wherein the one or combination of histatin peptides comprises a native histatin, synthetic histatin, or a combination thereof.

4. The method of claim 3, wherein said native histatin or synthetic histatin is linear or cyclized.

5. The method of claim 3, wherein the native histatin or synthetic histatin comprises a modification selected from glycosylation, acetylation, amidation, formylation, hydroxylation, methylation, myristoylation, phosphorylation, sulfonation, PEGylation or lipidation.

6. The method of claim 1, wherein the one or combination of histatin peptides are formulated for topical, oral, ocular, intravenous, intravitreal, subconjunctival, subcutaneous, intramuscular, intraperitoneal, intracerebral, intraarterial, intraportal, intralesional, intrathecal, or intranasal administration.

7. The method of claim 6, wherein the formulation is in the form of a gel, wash, cream, tablet, capsule, pill, solution, eye drop, spray, bandage, contact lens, depot, injectable, implantable, sustained-release or microparticle or nanoparticle formulation.

8. The method of claim 1, wherein the lysosomal storage disease is a glycogen storage disease, mucopolysaccaridosis, mucolipidosis, oligosaccharidosis, lipidosis, sphingolipidosis, or lysosomal transport disease.

9. The method of claim 8, wherein the lipidosis is Niemann-Pick type C disease.

10. A method for reducing accumulation of cholesterol, modulating calcium signaling, decreasing apoptotic signaling, reducing losses in cell viability, or reducing microtubule-associated protein 1 light chain 3 protein comprising administering to a subject in need of treatment an effective amount of one or a combination of histatin peptides to reduce accumulation of cholesterol, modulate calcium signaling, decrease apoptotic signaling, reduce losses in cell viability, or reduce microtubule-associated protein 1 light chain 3 protein accumulation.

11. The method of claim 10, wherein the cholesterol accumulation, calcium signaling, or microtubule-associated protein 1 light chain 3 accumulation is mediated by Niemann-Pick C protein deficiency.

12. A method for treating an ocular disease or condition comprising administering to a subject in need of treatment and effective amount of a TMEM97 modulator to treat the ocular disease or condition.

13. The method of claim 12, wherein the effective amount promotes wound healing and epithelial cell migration promoting activity in ocular tissue.
